(19) United States
(12) Patent Application Publication
(10) Pub. No.: US 2010/0310474 A1
(43) Pub. Date: Dec. 9, 2010

(54) PROTEIN CAGES FOR THE DELIVERY OF MEDICAL IMAGING AND THERAPEUTIC AGENTS

(75) Inventors: Mark J. YOUNG, Bozeman, MT (US); Trevor Douglas, Bozeman, MT (US); Yves U. Idzerda, Bozeman, MT (US)

Correspondence Address: COOLEY LLP;ATTN: Patent Group, Suite 1100, 777 - 6th Street, NW, WASHINGTON, DC, 20001, US

(73) Assignee: MONTANA STATE UNIVERSITY, Bozeman, MT (US)

(21) Appl. No.: 12/752,905

(22) Filed: Apr. 1, 2010

Related U.S. Application Data
(63) Continuation of application No. 11/415,485, filed on Apr. 27, 2006, now abandoned.
Application No. 11/415,485 is a division of application No. 10/441,962, filed on May 19, 2003, now abandoned.
(60) Provisional application No. 60/380,942, filed on May 17, 2002.

(51) Int. Cl. A61K 49/22; A61K 49/18; A61K 49/04; A61K 49/00; C12N 5/071; A61P 35/00

(52) U.S. Cl. 424/9.323; 424/9.1; 424/9.34; 424/9.4; 424/9.5; 424/9.32; 424/9.42; 424/9.322; 435/325

(57) ABSTRACT

The present invention is directed to novel compositions and methods utilizing delivery agents comprising protein cages, medical imaging agents and therapeutic agents.

### CROSS REFERENCE TO RELATED APPLICATIONS

This application is a divisional application of U.S. application Ser. No. 10/441,962, filed May 19, 2003, which claims the benefit of the filing date of Ser. No. 60/380,942, filed on May 17, 2002 under 35 U.S.C. § 119(e), which is expressly incorporated by reference in its entirety.

GOVERNMENTAL SUPPORT OF APPLICATION

This invention was made with governmental support under grant number GM61340, awarded by the National Institutes of Health. The United States government has certain rights in the invention.

### FIELD OF THE INVENTION

The present invention is directed to novel compositions and methods utilizing delivery agents comprising protein cages, medical imaging agents and therapeutic agents.

### BACKGROUND OF THE INVENTION

There is considerable interest in the chemical design and construction of self-assembling systems that can be used as delivery vehicles for encapsulated “guest” molecules. For example, viral capsid proteins are able to self assemble into highly symmetrical structures in a wide range of sizes from a very small number of basic building blocks (W. Chiu, R. Burnett, and R. Garcea (eds) (1997) “Structural Biology of Viruses Oxford”, University, N.Y.). In addition, viral capsids have evolved to encapsulate nucleic acids for protection, transport, and delivery to appropriate cells. Thus, in chemical terms, viral capsids can be viewed as playing molecular hosts to their nucleic acid guests. This host-guest property is of considerable interest to chemists taking a synthetic approach to making molecular cage-like structures, pioneered by the Nobel prizewinner Professor Donald Cram (“Container molecules and their guests” (1994) Royal Society of Chemistry, Cambridge). Host systems are characterized by clearly defined interiors and exteriors, i.e. interfaces that interact preferentially with the guests (interior) and with the bulk medium (exterior). The interior and exterior interfaces are chemically and geometrically different and it is these differences which provide specificity and function to the host (Cram, supra; Kang, J., and J. J. Rebek, 1997, Nature 385:50-52; and, Sherman, J. C., and D. J. Cram, 1989, J. Am. Chem. Soc. 111:4527-4528). The guest on the other hand has properties which allow it to interact specifically with the interior interface of the host. This molecular recognition is usually dependent on weak H-bonding, van der Waal's, and/or electrostatic interactions (Rebek, J., 1996, Chem. Soc. Rev. 25:255-264).

The capsid structures of viruses are a near perfect example of a highly evolved host-guest system functioning to store, transport, and release viral genomes and associated proteins. Capsids come in two basic geometric shapes: roughly spherical (usually based on icosahedral symmetry) and rod shaped (usually based on helical symmetry). All capsids have curvature which defines the overall size and shape of the host. Many viruses are pleomorphic and are able to assemble in a range of geometric configurations (icosahedrons, flat sheets, tubes etc.). In addition, many capsid structures of viruses undergo reversible structural transitions that play a role in the packaging or release of their nucleic acid ‘guests’.

There is high degree of structural similarity between the basic building blocks of many icosahedral viruses regardless of whether they infect animals, plants, insects, fungi or bacteria (Rossman, M. G., and J. E. Johnson, 1989, Annu. Rev. Biochem. 58:533-573). Most of the viral coat protein subunits have the β-barrel motif and assemble into hexameric and pentameric capsomer units. The ratio of hexamers to pentamers determines the curvature of the overall structure and ultimately the size of the final virion (Johnson, J. E., 1996, Proc. Natl. Acad. Sci. USA 93:27-33; Johnson, J. E., and J. A. Speir, 1998, J. Mol. Bio. 269:665-675; and Rossman and Johnson, 1989, supra). This is geometrically very similar to the assembly of fullerenes such as the so-called “buckyballs” (Smalley, R. E., 1992, Acc. Chem. Res. 25:98-105). The higher the hexamer to pentamer ratio, the larger the diameter of the structure. Spherical viruses typically range in size from 18-500 nm, while rod shaped viruses of >900 nm are known. At least conceptually, the natural variation in virus particle size and shape provides a wealth of potential protein cages.

The small spherical virus cowpea chlorotic mottle virus (CCMV) is an ideal model system for developing viral protein cages for cell-targeted bioimaging and therapeutic delivery. CCMV is a member of the bromovirus group of the Bromoviridae (a member of the alpha family supergroup) (Ahlquist, P., 1992, Curr. Opin. Gen. and Dev. 2:71-76; Dasgupta, R., and P. Kaesberg, 1982, Nucleic Acid Res. 5:987-998; and Lane, L. C., 1981, The Bromoviruses. In E. Kurstak (ed.), “Handbook of plant virus infection and comparative diagnosis”, Elsevier/North-Holland, Amsterdam). Bromovirues are 25-28 nm icosahedral viruses with a four component (+) sense single stranded RNA genome. CCMV has been used as a model system for viral assembly since 1967 when Bancroft and Hiebert demonstrated that purified RNA and coat protein self-assemble in vitro to produce infectious virions (Bancroft, J. B., et al., 1969, Virology 38:324-335; Bancroft, J. B., and E. Hiebert, 1967, Virology 32:354-356; Bancroft, J. B., et al., 1968, Virology 36:146-149; Hiebert, E., and J. B. Bancroft, 1969, Virology 39:296-311; and Hiebert, E., et al., 1968, Virology 34:492-508).

Other protein cages that may be useful for cell-targeted bioimaging and therapeutic delivery include apoferritin and the heat shock protein from Methanococcus jannaschii. See for example, Douglas et al., 1995, “Inorganic-Protein Interactions in the Synthesis of a Ferromagnetic Nanocomposite,” American Chemical Society, ACS Symposium Series: Hybrid Organic-Inorganic Composites, J. Mark, C. Y -C Lee, P. A. Bianconi (eds.); Douglas et al., 1995, Science 269: 54-57; Bulte et al., May/June 1994, JMRI pp. 497-505; Meldrum et al., 1992, Science 257: 522-523; Bulte et al., 1995, Acad. Radiol. 2: 871-878; and, Bulte et al., 1994, Investigative Radiobiology 20 (Suppl. 2): S214-5216 for apoferritin cages. See for example, Kim, K. K. et al., 1998, Nature 394:595-599; Kim, K. K. et al., 1998, J. Struct. Biol. 121:76-80; and Kim, K. K. et al., 1998, PNAS 95:9129-9133 for the heat shock protein of Methanococcus jannaschii.

Accordingly, it is an object of the present invention to provide novel compositions and uses for protein cages as delivery agents for medical imaging and therapeutic agents.

### SUMMARY OF THE INVENTION

The present invention provides compositions, methods for making and uses for delivery agents comprising protein cages loaded with at least one medical imaging agent, and preferably at least one therapeutic agent. Preferred embodiments utilize empty virion protein cages. The compositions and methods employ unmodified and modified protein cages that can be loaded (loaded includes the synthesis of materials within the cage) with various combinations of medical imaging and therapeutic agents. Loading of the medical imaging and therapeutic agents may be facilitated through the use of attachment linkers, such as polymers and homo-or hetero-bifunctional linkers.

In one embodiment, at least one medical imaging agent is introduced into the protein cage by triggering a reversible structural change in the protein cage. Preferably, a chemical switch is used to shift the cage from a closed form to an open form. In the open form, soluble material can be freely exchanged between the inside and outside of the protein cage. Shifting the cage back to the closed form results in the entrapment of the soluble material inside of the cage. In this manner, a large number of soluble medical imaging agents and/or therapeutic agents may be introduced into the cage's interior. Subsequent triggering of the chemical switch results in the release of the agents at a cell, tissue or organ of interest.

In other embodiments, modified protein cages are loaded with at least one medical imaging agent, and in some embodiments, preferably at least one therapeutic agent. The modifications include the engineering of new chemical switches that are redox-sensitive or pH sensitive. In addition, the cage can be modified to provide for the incorporation of a targeting moiety.

The compositions and methods of the present invention provide significant advantages over currently available delivery agents, such as liposomes. By virtue of their high loading capacity, a large number of introduced molecules can be packaged within the cage. Moreover, the protein cage can function as a constrained reaction vessel facilitating the aggregation and crystallization of introduced molecules. Other advantages include the ability to control the size of the cage and cage components, and extend the range of imaging and therapeutic delivered through chemical and genetic modifications to the cage.

The compositions and methods of the invention find use in myriad applications for bioimaging and delivery of a therapeutic agent to a cell or tissue of interest. As specific non-limiting examples, the compositions and methods may be used to obtain an image of a cell, tissue or patient and/or introduce a therapeutic agent to a diseased tissue or organ of interest.

### DETAILED DESCRIPTION OF THE DRAWINGS

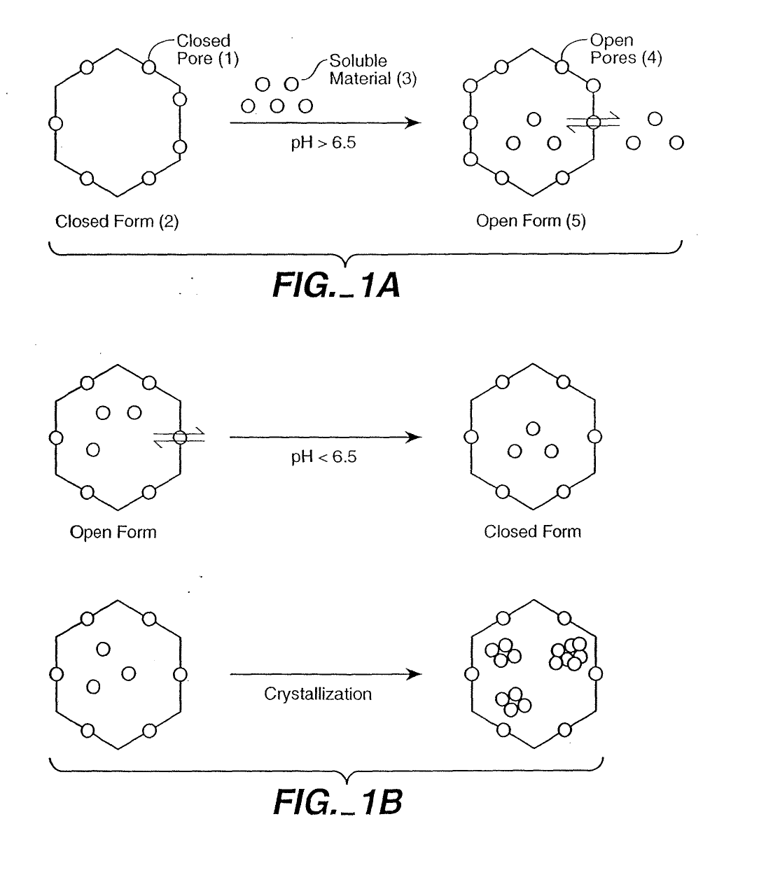

FIG. 1 illustrates the basic principle of introducing soluble material such as a medical imaging agent into a protein cage;

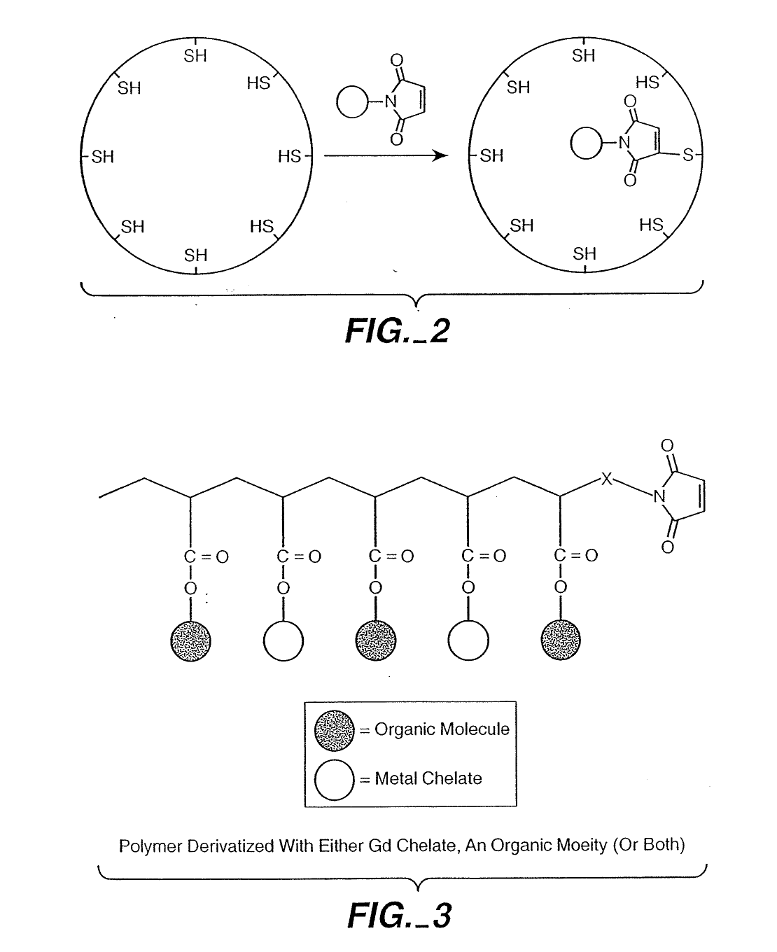

FIG. 2 illustrates a protein cage that has been modified to introduce a new chemical switch via the addition of cysteine residues on the inside of the cage;

FIG. 3 illustrates the introduction of a large anionic organic polymer using a pH chemical switch;

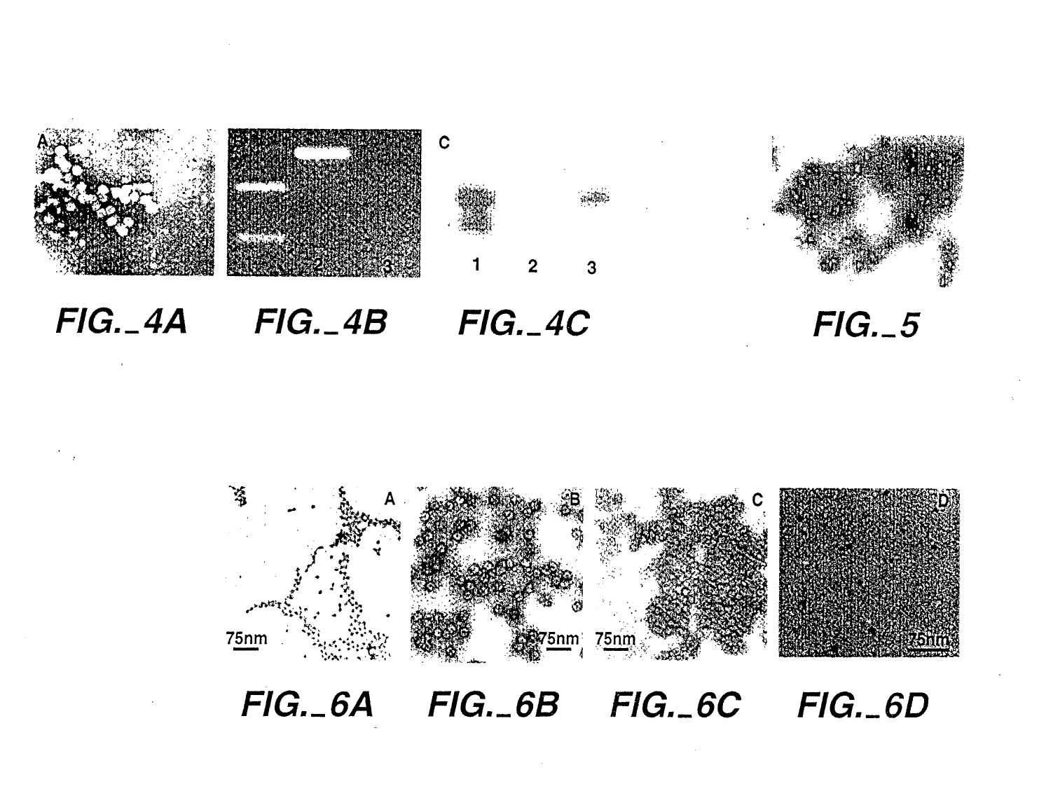

FIG. 4A-C illustrates the expression of a targeting moiety on the exterior surface of a

CCMV protein cage. FIG. 4A is a TEM of peptide 11 protein cages from the P. pastoris system. FIG. 4B is a PCR digest of coat protein. Lane 1, protein cage with peptide 11 protein; lane 2, wild type coat protein, and lane 3, no peptide 11 control. FIG. 4C is a westron blot of P. pastoris expressing peptide 11 coat protein (lane 1); wild type coat protein (lane 2) and control (lane 3);

FIG. 5 illustrates the production of CCMV protein cages in a yeast-based heterologous protein expression system; and

FIG. 6A-D illustrates examples of different materials entrapped/crystallized within the CCMV protein cage. FIG. 6A is an unstained sample of H₂WO₄₂¹⁰⁻ cores. FIG. 6B is a negative stain sample of H₂WO₄₂¹⁰⁻ cores showing protein cages. FIG. 6C is a negative sample of encapsulated polyanetholesulphonic acid. FIG. 6D is an unstained sample of ferric oxide cores in P. pastoris expressed protein cages.

### DETAILED DESCRIPTION

The present invention is directed to the discovery that protein cages can be used as constrained reaction vessels for the selective entrapment and release of materials. A unique aspect of protein cages that makes them attractive as delivery vehicles is there ability to undergo reversible structural changes allowing for the formation of open pores through which material can pass. These reversible changes can be controlled by factors such as pH and ionic strength. For example, pH can be used to control the expansion and contraction of the protein cage (see FIG. 1A-C). When the cage is expanded, i.e., opened, pores are formed allowing for the free exchange of soluble material between the inside and outside of the cage (see FIG. 1A). When the cage is contracted, i.e., closed, the pores are closed and any material in the cage is trapped within (see FIG. 1B). In some embodiments, material trapped within the cage can undergo crystallization, thereby increasing the quantity of material within the cage. The cage can then be isolated as a crystal containing nano-composite. As this process is freely reversible process, the material can be released by placing the cage under conditions that allow for the expansion of the cage and the formation of open pores (see FIG. 1C). This approach is borrowed from the synthesis of nano-phase inorganic materials from solution and applies equally well to inorganic and organic species.

Previous work has utilized several different types of protein “shells” that can be loaded with different types of materials. For example, as outlined above and herein, virion nanoparticles comprising a shell of coat protein(s) that encapsulate a non-viral material have been described; see U.S. Pat. No. 6,180,389, hereby incorporated by reference in its entirety. Similarly, as described above mammalian apoferritin protein cages have been loaded with various materials.

Additionally, protein cages can be modified to alter the factors that control the opening and closing of the cage. For example, amino acid residues may be substituted for existing amino acid residues to alter the pH sensitivity and redox sensitivity. Other modifications include the expression of heterologous amino acid sequences on surface of the cage that can then be used to direct, i.e., target the cage, to a particular location in a cell, tissue or organ.

Accordingly, the present invention is directed to the use of protein cages as delivery vehicles for various biomedical applications. The cages can be loaded with any number of different materials, including organic, inorganic, and metallorganic materials, and mixtures thereof. Particularly preferred embodiments utilize combinations of medical imaging agents and therapeutic agents for use as imaging and therapeutic agents.

One advantage of the present invention is that a combination of medical imaging agents can be loaded into the cage. For example imaging agents for magnetic resonance imaging and x-ray imaging can be combined in one cage thereby allowing the resulting agent to be used with a multiple imaging methods. Another substantial advantage over the prior art is that protein cages are capable of encapsulating a larger number of molecules than other vehicles, i.e. liposomes, commonly used for the delivery of therapeutic agents. For example, up to 29,600 molecules of H₂WO₄₂¹⁰⁻ have been packaged as a nano size crystalline solid within the cowpea chlorotic mottle virus (CCMV) protein cage. The size and shape of the crystallized nano material is determined by the size and shape of the cavity created by the CCMV protein cage. One other advantage, is that the protein cage can be used to increase the number of introduced materials present in the interior of the cage via crystallization. The crystallization of introduced materials can controlled because the protein cage provides a charged protein interface (on the interior) which can facilitate the aggregation and crystallization of ions.

Accordingly, the present invention provides compositions comprising a plurality of delivery agents. By “delivery agent” herein is meant a proteinaceous shell that self-assembles to form a protein cage (e.g. a structure with an interior cavity which is either naturally accessible to a solvent or can be made to be so by altering solvent concentration, pH, equilibria ratios, etc.), and contains imaging and therapeutic agents as discussed below. The protein cage may be obtained from a non-viral or viral source.

Preferred non-viral protein cages include ferritins and apoferritins, both eukaryotic and prokaryotic species, in particular mammalian and bacteria, with 12 and 24 subunit ferritins being especially preferred. In addition, 24 subunit heat shock proteins forming an internal core space are included. In particular, the heat shock protein of Methanococcus jannaschii assembles into a 24 subunit cage with 432 symmetry (see Kim, K. K. et al., 1998, Nature 394:595-599; Kim, K. K. et al., 1998, J. Struct. Biol. 121:76-80; and Kim, K. K. et al., 1998, PNAS 95:9129-9133).

Preferred viral protein cages can be obtained from any animal or plant virus from which empty viral particles can be produced. For example, empty viral particle can be obtained from viruses belonging to the bromovirus group of the Bromoviridae (Ahlquist, P., 1992, Curr. Opin. Gen. and Dev. 2:71-76; Dasgupta, R., and P. Kaesberg, 1982, Nucleic Acid Res. 5:987-998; and Lane, L. C., 1981, The Bromoviruses. In E. Kurstak (ed.), “Handbook of plant virus infection and comparative diagnosis”, Elsevier/North-Holland, Amsterdam) and from the family Caliciviridae. Viruses suitable for use in the invention include cowpea chlorotic mottle virus (CCMV) and the Norwalk virus.

In a preferred embodiment, empty viral particles are obtained from CCMV. A 3.2 Å resolution structure of CCMV is available that can be used to predict the role of individual amino acids in controlling virion assembly, stability , and disassembly (Speir, J. A., et al., 1995, Structure 3:63-78). The virion is made up of 180 copies of the coat protein subunit arranged with a T=3 quasi-symmetry and organized in 20 hexamer and 12 pentameric capsomers. A striking feature of the coat protein subunit is the presence of N- and C-terminal ‘arms’ that extend away from the central, eight-stranded, antiparallel b-barrel core. Each coat protein consists of a canonical β-barrel fold (formed by amino acids 52-176) from which long N-terminal (residues 1-51; 1-27 are not ordered in the crystal structure) and C-terminal arms (residues 176-190) extend in opposite directions. These N- and C-terminal arms provide an intricate network of ‘ropes’ which ‘tie’ subunits together. The first 25 amino acids are found lining the interior surface of the virion (Rao, A. L. and G. L. Grantham, 1996, Virology 226:294-305; and, Zhao, X., et al., 1995, Virology, 207:486-494). These 25 amino acids are thought to be highly mobile and to be required for viral RNA packaging. Nine of the first 25 amino acids are basic (Arg, Lys) and are thought to neutralize the negatively charged RNA. The first 25 amino acids are not required for empty virion assembly (devoid of viral RNA) and thus can be modified to change the electrostatic nature of the virion's interior surface, etc. The orientation of the coat protein β-barrel fold is nearly parallel to the five-fold and quasi six-fold axes. This orientation results in five exterior surface-exposed loops, βB-βC, βD-βE, βF-βG, βC-αCD1, βH-βI. Surrounding each of the 60 quasi three-fold axes located on the interface between hexamer and pentamer capsomers are Ca²⁺ binding sites. There are 180 Ca²⁺ binding sites per virion. Each Ca²⁺ binding site consists of five residues (Glu81, Gln85, Glu148 from one subunit; Gln 149 and Asp 153 from an adjacent subunit) in an ideal position to coordinate Ca²⁺ binding.

The protein cage may be unmodified or modified. By “unmodified” or “native” herein is meant a protein cage that has not been genetically altered or modified by other physical, chemical or biochemical means. By “modified” or “altered” herein is meant a protein cage that has been genetically altered or modified by a physical, chemical or biochemical means.

In a preferred embodiment, the protein cage is modified. Preferably, the modification results in protein cages with improved properties for use as delivery vehicles. For example, protein cages can be designed that are more stable than the unmodified cages or to contain binding sites for metal ions. Additionally, protein cages can be designed that have different charged interior surfaces for the selective entrapment and aggregation of medical imaging or therapeutic agents. Other modifications include the introduction of new chemical switches that can be controlled by pH or by redox conditions, the introduction of targeting moieties on the exterior surface, the addition of functional groups for the subsequent attachment of additional moieties, and covalent modifications.

In a preferred embodiment, protein cages are genetically modified to be more stable. Native CCMV virions are stable over a broad pH range (pH 2-8) and temperature (−80 to 72° C.) (Zhao, X., et al., 1995, Virology, 207:486-494). Empty virions (assembled CCMV protein cages) are stable over this range when assembled from mutants of the coat protein. The salt stable coat protein mutation (K42R) (Fox, J. M., et al., 1996, Virology 222:115-122) and the cysteinyl mutation (R26C) (Fox, J., et al., 1997, Virology 227:229-233.32) both result in empty virions that are stable over this broad pH and temperature range.

In a preferred embodiment, protein cages are modified by the introduction of functional groups on the inside of the protein cage. In one embodiment, ion binding sites in the interior of the cage are modified to bind paramagnetic metals, such as gadolinium (Gd(III) or Gd³⁺). Preferably, existing Ca ²⁺ binding sites are modified to enhance binding of Gd(III) (see Example 1). Preferably, through the use and modification of existing metal binding sites from 1 to 180 Gd(III) ions can be incorporated per cage. More preferably, cages may comprise the following ranges of Gd(III) ions: 10 to 180, 50 to 180, 75 to 180, 100 to 180 and 150 to 180 Gd(III) ions.

In a preferred embodiment, protein cages are modified to provide an interface for molecular aggregation, i.e., crystallization, based on complementary electrostatic interactions between the protein cage and the entrapped material. Previous work has shown that protein cages are ideal reaction vessels for the constrained crystallization of guest molecules (Douglas, T., and M. J. Young, 1998, Nature 393:152-155). For example, a range of polyoxometalate species (i.e., vanadate, molybdate, tungstate) have been crystallized within CCMV protein cages. Similarly, tungstate has been crystallized with the Norwalk Virus protein cage (see Example 2).

In a preferred embodiment, the N-terminal arm of the coat protein subunit of CCMV is genetically modified to aggregate new classes of materials including the so-called soft metals, including Fe(II) (Cotton, F. A., and G. Wilkinson., 1999, Inorganic Chemistry, John Wiley & Sons), polyanions (i.e. poly(dextran sulfate) and poly (anetholosulfonic acid)), small molecules (i.e., drug and drug analogs), polycationic species (i.e. poly(ethylenimine), poly(lysine), poly(arginine) and poly(vinylimidazoline) (see Example 2).

In a preferred embodiment, protein cages are modified to provide improved or new chemical switching or gating mechanisms, i.e. chemical switches, that control the reversible swelling of the cages. For example, many viruses are known to undergo reversible structural transitions. The reversible swelling of the CCMV virion is one of the most thoroughly characterized of these structural transitions (Fox, J. M., et al., 1996, Virology 222:115-12235; and Speir, J. A., et al., 1995, Structure. 3:63-78). At pH values <6.5 the virion exists in its compact or closed form. Increasing the pH above 6.5, in the absence of Ca²⁺, results in an 10% expansion (swelling) in the overall dimensions of the virion. Modeling of the CCMV X-ray crystal structure, combined with cryo electron microscopy and image reconstruction of swollen CCMV, indicates that virion swelling is a result of expansion at the quasi three-fold axis of the virion (Speir, J. A., et al., 1995, Structure. 3:63-78). Swelling results in the creation of sixty 20 Å holes which provide access between the interior and exterior of the virion. Thus, one can think of pH as a chemical switch for controlling access to and from the central cavity of the CCMV protein cage (virion). Other means for controlling access to and from the central cavity of protein cages, include redox conditions.

In a preferred embodiment, protein cages are modified to provide improved or new chemical switches for the introduction and delivery of imaging and therapeutic agents. By “chemical switch” herein is meant a factor present in the microenvironment of the protein cage that can be used to control the access to and from the cage's interior. As will be appreciated by those of skill in the art, the switches may be reversible or irreversible (i.e. suicide switches).

By “reversible” herein is a meant a switch that can function in both directions. That is, the switch can be activated to open and close the pores of the cage to allow passage of material in and out of the cage. Examples of chemical switches include pH, ionic strength of the medium, redox conditions, etc.

In a preferred embodiment, protein cages are modified to introduce reversible pH activated switches. Preferably, the pH sensitive switch is an acid sensitive switch. Acid sensitive switches may be introduced by adding histidine residues at the Ca²⁺ binding site (see Example 3).

In a preferred embodiment, protein cages are modified to introduce reversible redox activated switches. Redox sensitive switches may be introduced by cysteine residues near the Ca²⁺ binding site (see Example 3).

In a preferred embodiment, the protein cages are modified to provide irreversible or suicide switches. By “irreversible” or “suicide switches” herein is meant switches that operate in only one direction. In other words, these switches are activated to allow either the entry or exit of materials, but not both. Examples of irreversible switches that may introduced into protein cages include pH switches, redox switches, radiation induction heating switches, near IR switches, radiation induced disassembly, protease sensitive switches and metal dependent switches.

In a preferred embodiment, protein cages are modified to allow for the attachment of functional groups that can be used to attach imaging and therapeutic agents. For example replacement of amino acids on the inner surface of the cage by cysteine residues results in the presentation of reactive —SH groups on the inner surface. In addition to the role of —SH groups in redox activated switching, —SH groups can be reactive with bifunctional agents, such as maleimide to attach diagnostic agents (i.e. MRI imaging agents) and therapeutic agents to the interior of the cage (see FIG. 2).

In a preferred embodiment, protein cages are modified for the attachment of targeting moieties. By the term “targeting moiety” herein is meant a functional group that serves to target or direct the delivery vehicle, i.e., the cage comprising at least one medical imaging agent, to a particular location or association, i.e. a specific binding event. Thus, for example, a targeting moiety may be used to target a molecule to a specific target protein or enzyme, or to a particular cellular location, to a particular cell type, to a diseased tissue. As will be appreciated by those in the art, the localization of proteins within a cell is a simple method for increasing effective concentration. For example, shuttling an imaging agent and/or drug into the nucleus confines them to a smaller space thereby increasing concentration. Finally, the physiological target may simply be localized to a specific compartment, and the agents must be localized appropriately.

Suitable targeting moieties include, but are not limited to, proteins, nucleic acids, carbohydrates, lipids, hormones including proteinaceous and steroid hormones, growth factors, receptor ligands, antigens and antibodies, and the like. Proteins in this context means proteins (including antibodies), oligopeptides and peptides, derivatives and analogs, including proteins containing non-naturally occurring amino acids and amino acid analogs, and peptidomimetic structures. The side chains may be in either the (R) or the (S) configuration. In a preferred embodiment, the amino acids are in the (S) or L-configuration.

Suitable targeting sequences include, but are not limited to, binding sequences capable of causing binding of the moiety to a predetermined molecule or class of molecules while retaining bioactivity of the expression product, (for example by using enzyme inhibitor or substrate sequences to target a class of relevant enzymes); sequences signaling selective degradation, of itself or co-bound proteins; and signal sequences capable of constitutively localizing the candidate expression products to a predetermined cellular locale, including a) subcellular locations such as the Golgi, endoplasmic reticulum, nucleus, nucleoli, nuclear membrane, mitochondria, chloroplast, secretory vesicles, lysosome, and cellular membrane; and b) extracellular locations via a secretory signal. Particularly preferred are proteins, including peptides, antibodies and cell surface ligands.

In a preferred embodiment, the targeting moiety is a peptide. For example, chemotactic peptides have been used to image tissue injury and inflammation, particularly by bacterial infection; see WO 97/14443, hereby expressly incorporated by reference in its entirety.

In a preferred embodiment, the targeting moiety is laminin peptide 11. Peptide 11 is a well characterized system for understanding cancer cell metastasis (Landowski, T. H., et al., 1995, Biochemistry, 34:11276-11287; Landowski, T. L., et al., 1995, Clin. Exp. Metastasis 13:357-372; Menard, S., et al., 1997, J. Cell Biochem. 67:155-165; J. R. Starkey, et al., 1999, Cytometry 35:37-47; Starkey, J. R., 1994, Human Pathology, 25:1259-1260; and, J. R. Starkey, et al., 1998, Biochim. Biophys. Acta. 1429:187-207). Briefly outlined, the interactions of tumor cells with basement membrane components are considered to be critical determinants of the ability of a tumor to invade and spread to distant sites. The 67 kDa high affinity laminin binding protein (LBP) is a cell surface protein thought to mediate such invasive interactions (Menard, S., et al., 1997, J. Cell Biochem. 67:155-165). The expression of LBP is positively correlated with progression in many solid tumors (Mafune, K., et al., 1990, Cancer Res. 50:3888-3891; Sanjuan, X., et al., 1996, J. Pathol. 179:376-380; and Viacava, P., et al., 1997, J. Pathol. 182:36-44). The major ligand binding for LBP is the laminin-1 protein. A ten amino acid sequence from laminin-1b chain, CDPGYIGSRC, known as peptide 11, is the primary ligand binding domain for LBP (Graf, J., et al., 1987, Cell, 48:989-996; Iwamoto, Y., et al., 1996, Br. J. Cancer 73:589-595; and Iwamoto, Y., et al., 1987, Science 238:1132-1134). Free peptide 11 effectively blocks invasion of basement membranes by tumor cells, reduces experimental tumor lung colonization, and inhibits tumor angiogenesis in mice (Mafune, K., et al., 1990, Cancer Res. 50:3888-3891; Sanjuan, X. et al., 1996, J. Pathol. 179:376-380; and Viacava, P., et al., 1997, J. Pathol. 182:36-44). Research, using both in vitro binding assays and in situ localization studies, has strongly suggested that the anti-metastatic activity of free peptide 11 is a direct result of binding to the LBP (J. R. Starkey, et al., 1999, Cytometry 35:37-47; and J. R. Starkey, et al., 1998, Biochim. Biophys. Acta. 1429:187-207). Among the known sequences for animal proteins, the peptide 11 sequence is specific to laminin. The 67 kDa LBP is highly conserved in evolution (Bignon, C., et al., 1992, Biochem Biophys Res Commun. 184:1165-1172) and has been shown to be expressed quite early in development where it likely plays a role in the direction of cell migration on laminin containing substrates (Laurie, G. W., et al., 1989, J. Cell Biol. 109:1351-1362). The 67 kDa LBP is also present on platlets and neutrophils, but free peptide 11 has no toxic effect on these cells. The properties of peptide 11 suggest that it could be adapted for tumor targeting. As described in Example 4, we have expressed peptide 11 on the surface of the CCMV protein cage in an effort to target these cages to solid tumor cells.

In a preferred embodiment, the targeting moiety is an antibody. The term “antibody” includes antibody fragments, as are known in the art, including Fab Fab₂, single chain antibodies (Fv for example), chimeric antibodies, etc., either produced by the modification of whole antibodies or those synthesized de novo using recombinant DNA technologies.

In a preferred embodiment, the antibody targeting moieties of the invention are humanized antibodies or human antibodies. Humanized forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature 321:522-525 (1986); Riechmann et al., Nature 332:323-329 (1988); and Presta, Curr. Op. Struct. Biol. 2:593-596 (1992)].

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers [Jones et al., Nature 321:522-525 (1986); Riechmann et al., Nature 332:323-327 (1988); Verhoeyen et al., Science 239:1534-1536 (1988)], by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol. 227:381 (1991); Marks et al., J. Mol. Biol. 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol. 147(1):86-95 (1991)]. Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology, 14:826 (1996); Lonberg and Huszar, Intern. Rev. Immunol. 13:65-93 (1995).

Bispecific antibodies are monoclonal, preferably human or humanized, antibodies that have binding specificities for at least two different antigens. In the present case, one of the binding specificities is for a first target molecule and the other one is for a second target molecule.

Methods for making bispecific antibodies are known in the art. Traditionally, the recombinant production of bispecific antibodies is based on the co-expression of two immunoglobulin heavy-chain/light-chain pairs, where the two heavy chains have different specificities [Milstein and Cuello, Nature 305:537-539 (1983)]. Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., EMBO J. 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology 121:210 (1986).

Heteroconjugate antibodies are also within the scope of the present invention. Heteroconjugate antibodies are composed of two covalently joined antibodies. Such antibodies have, for example, been proposed to target immune system cells to unwanted cells [U.S. Pat. No. 4,676,980], and for treatment of HIV infection [WO 91/00360; WO 92/200373; EP 03089]. It is contemplated that the antibodies may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

In a preferred embodiment, the antibody is directed against a cell-surface marker on a cancer cell; that is, the target molecule is a cell surface molecule. As is known in the art, there are a wide variety of antibodies known to be differentially expressed on tumor cells, including, but not limited to, HER2, VEGF, etc.

In addition, antibodies against physiologically relevant carbohydrates may be used, including, but not limited to, antibodies against markers for breast cancer (CA15-3, CA 549, CA 27.29), mucin-like carcinoma associated antigen (MCA), ovarian cancer (CA125), pancreatic cancer (DE-PAN-2), and colorectal and pancreatic cancer (CA 19, CA 50, CA242).

In one embodiment, antibodies against virus or bacteria can be used as targeting moieties. As will be appreciated by those in the art, antibodies to any number of viruses (including orthomyxoviruses, (e.g. influenza virus), paramyxoviruses (e.g respiratory syncytial virus, mumps virus, measles virus), adenoviruses, rhinoviruses, coronaviruses, reoviruses, togaviruses (e.g. rubella virus), parvoviruses, poxviruses (e.g. variola virus, vaccinia virus), enteroviruses (e.g. poliovirus, coxsackievirus), hepatitis viruses (including A, B and C), herpesviruses (e.g. Herpes simplex virus, varicella-zoster virus, cytomegalovirus, Epstein-Barr virus), rotaviruses, Norwalk viruses, hantavirus, arenavirus, rhabdovirus (e.g. rabies virus), retroviruses (including HIV, HTLV-I and -II), papovaviruses (e.g. papillomavirus), polyomaviruses, and picornaviruses, and the like), and bacteria (including a wide variety of pathogenic and non-pathogenic prokaryotes of interest including Bacillus; Vibrio, e.g. V. cholerae; Escherichia, e.g. Enterotoxigenic E. coli, Shigella, e.g. S. dysenteriae; Salmonella, e.g. S. typhi; Mycobacterium e.g. M. tuberculosis, M. leprae; Clostridium, e.g. C. botulinum, C. tetani, C. difficile, C. perfringens; Cornyebacterium, e.g. C. diphtheriae; Streptococcus, S. pyogenes, S. pneumoniae; Staphylococcus, e.g. S. aureus; Haemophilus, e.g. H. influenzae; Neisseria, e.g. N. meningitidis, N. gonorrhoeae; Yersinia, e.g. G. lambliaY. pestis, Pseudomonas, e.g. P. aeruginosa, P. putida; Chlamydia, e.g. C. trachomatis; Bordetella, e.g. B. pertussis; Treponema, e.g. T. palladium; and the like) may be used.

In a preferred embodiment, the targeting moiety is all or a portion (e.g. a binding portion) of a ligand for a cell surface receptor. Suitable ligands include, but are not limited to, all or a functional portion of the ligands that bind to a cell surface receptor selected from the group consisting of insulin receptor (insulin), insulin-like growth factor receptor (including both IGF-1 and IGF-2), growth hormone receptor, glucose transporters (particularly GLUT 4 receptor), transferrin receptor (transferrin), epidermal growth factor receptor (EGF), low density lipoprotein receptor, high density lipoprotein receptor, leptin receptor, estrogen receptor (estrogen); interleukin receptors including IL-1, IL-2, IL-3, IL-4, IL-5, IL-6, IL-7, IL-8, IL-9, IL-11, IL-12, IL-13, IL-15, and IL-17 receptors, human growth hormone receptor, VEGF receptor (VEGF), PDGF receptor (PDGF), transforming growth factor receptor (including TGF-α and TGF-β), EPO receptor (EPO), TPO receptor (TPO), ciliary neurotrophic factor receptor, prolactin receptor, and T-cell receptors. In particular, hormone ligands are preferred. Hormones include both steroid hormones and proteinaceous hormones, including, but not limited to, epinephrine, thyroxine, oxytocin, insulin, thyroid-stimulating hormone, calcitonin, chorionic gonadotropin, cortictropin, follicle-stimulating hormone, glucagon, leuteinizing hormone, lipotropin, melanocyte-stimutating hormone, norepinephrine, parathryroid hormone, thyroid-stimulating hormone (TSH), vasopressin, enkephalins, seratonin, estradiol, progesterone, testosterone, cortisone, and glucocorticoids and the hormones listed above. Receptor ligands include ligands that bind to receptors such as cell surface receptors, which include hormones, lipids, proteins, glycoproteins, signal transducers, growth factors, cytokines, and others.

In a preferred embodiment, the targeting moiety is a carbohydrate. By “carbohydrate” herein is meant a compound with the general formula Cx(H2O)y. Monosaccharides, disaccharides, and oligo- or polysaccharides are all included within the definition and comprise polymers of various sugar molecules linked via glycosidic linkages. Particularly preferred carbohydrates are those that comprise all or part of the carbohydrate component of glycosylated proteins, including monomers and oligomers of galactose, mannose, fucose, galactosamine, (particularly N-acetylglucosamine), glucosamine, glucose and sialic acid, and in particular the glycosylation component that allows binding to certain receptors such as cell surface receptors. Other carbohydrates comprise monomers and polymers of glucose, ribose, lactose, raffinose, fructose, and other biologically significant carbohydrates. In particular, polysaccharides (including, but not limited to, arabinogalactan, gum arabic, mannan, etc.) have been used to deliver MRI agents into cells; see U.S. Pat. No. 5,554,386, hereby incorporated by reference in its entirety.

In a preferred embodiment, the targeting moiety is a lipid. “Lipid” as used herein includes fats, fatty oils, waxes, phospholipids, glycolipids, terpenes, fatty acids, and glycerides, particularly the triglycerides. Also included within the definition of lipids are the eicosanoids, steroids and sterols, some of which are also hormones, such as prostaglandins, opiates, and cholesterol.

In a preferred embodiment, the targeting moiety is all or a portion of the HIV-1 Tat protein, and analogs and related proteins, which allows very high uptake into target cells. See for example, Fawell et al., PNAS USA 91:664 (1994); Frankel et al., Cell 55:1189 (1988); Savion et al., J. Biol. Chem. 256:1149 (1981); Derossi et al., J. Biol. Chem. 269:10444 (1994); Baldin et al., EMBO J. 9:1511 (1990); Watson et al., Biochem. Pharmacol. 58:1521 (1999); Schwarze et al., TiPS (2000) 21:45; and Lindgren, TiPS 21:99 (2000); all of which are incorporated by reference.

In a preferred embodiment, the targeting moiety is a nuclear localization signal (NLS). NLSs are generally short, positively charged (basic) domains that serve to direct the moiety to which they are attached to the cell's nucleus. Numerous NLS amino acid sequences have been reported including single basic NLS's such as that of the SV40 (monkey virus) large T Antigen (Pro Lys Lys Lys Arg Lys Val), Kalderon (1984), et al., Cell, 39:499-509; the human retinoic acid receptor-β nuclear localization signal (ARRRRP); NFκB p50 (EEVQRKRQKL; Ghosh et al., Cell 62:1019 (1990); NFκB p65 (EEKRKRTYE; Nolan et al., Cell 64:961 (1991); and others (see for example Boulikas, J. Cell. Biochem. 55(1):32-58 (1994), hereby incorporated by reference) and double basic NLS's exemplified by that of the Xenopus (African clawed toad) protein, nucleoplasmin (Ala Val Lys Arg Pro Ala Ala Thr Lys Lys Ala Gly Gln Ala Lys Lys Lys Lys Leu Asp), Dingwall, et al., Cell, 30:449-458, 1982 and Dingwall, et al., J. Cell Biol., 107:641-849; 1988). Numerous localization studies have demonstrated that NLSs incorporated in synthetic peptides or grafted onto reporter proteins not normally targeted to the cell nucleus cause these peptides and reporter proteins to be concentrated in the nucleus. See, for example, Dingwall, and Laskey, Ann, Rev. Cell Biol., 2:367-390, 1986; Bonnerot, et al., Proc. Natl. Acad. Sci. USA, 84:6795-6799, 1987; Galileo, et al., Proc. Natl. Acad. Sci. USA, 87:458-462, 1990.

In a preferred embodiment, targeting moieties for the hepatobiliary system are used; see U.S. Pat. Nos. 5,573,752 and 5,582,814, both of which are hereby incorporated by reference in their entirety.

Targeting moieties may be added to the surface of protein cages either by engineering protein cages to express the targeting moiety or by the addition of functional groups to the surface of the protein cage. In a preferred embodiment, the protein cage is engineered to express the targeting moiety. For example, one or more of the five surface exposed loops may be used for the expression of the targeting moiety (see Example 4).

In a preferred embodiment, targeting moieties are add to the surface of protein cages through the use of functional groups. Functional groups may be added to the protein cage for subsequent attachment to additional moieties. Preferred functional groups for attachment are amino groups, carboxy groups, oxo groups and thiol groups. These functional groups can then be attached, either directly or indirectly through the use of a linker. Linkers are well known in the art; for example, homo-or hetero-bifunctional linkers as are well known (see 1994 Pierce Chemical Company catalog, technical section on cross-linkers, pages 155-200, as well as the 2003 catalog, both of which are incorporated herein by reference). Preferred linkers include, but are not limited to, alkyl groups (including substituted alkyl groups and alkyl groups containing heteroatom moieties), with short alkyl groups, esters, amide, amine, epoxy groups and ethylene glycol and derivatives being preferred, with propyl, acetylene, and C₂ alkene being especially preferred.

In preferred embodiments, covalent modifications of protein cages are included within the scope of this invention. One type of covalent modification includes reacting targeted amino acid residues of cage residue with an organic derivatizing agent that is capable of reacting with selected side chains or the N-or C-terminal residues of a cage polypeptide. Derivatization with bifunctional agents is useful, for instance, for crosslinking the cage to a water-insoluble support matrix or surface for use in the methods described below. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidyl propionate), bifunctional maleimides such as bis-N-maleimido-1,8-octane and agents such as methyl-3-[(p-azidophenyl)dithio]propioimidate. Crosslinking agents find particular use in 2 dimensional array embodiments.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the “-amino groups of lysine, arginine, and histidine side chains [T. E. Creighton, Proteins: Structure and Molecular Properties, W. H. Freeman & Co., San Francisco, pp. 79-86 (1983)], acetylation of the N-terminal amine, and amidation of any C-terminal carboxyl group.

Another type of covalent modification of cages, if appropriate, comprises altering the native glycosylation pattern of the polypeptide. “Altering the native glycosylation pattern” is intended for purposes herein to mean deleting one or more carbohydrate moieties found in native sequence of the cage monomer, and/or adding one or more glycosylation sites that are not present in the native sequence.

Addition of glycosylation sites to cage polypeptides may be accomplished by altering the amino acid sequence thereof. The alteration may be made, for example, by the addition of, or substitution by, one or more serine or threonine residues to the native sequence polypeptide (for O-linked glycosylation sites). The amino acid sequence may optionally be altered through changes at the DNA level, particularly by mutating the DNA encoding the polypeptide at preselected bases such that codons are generated that will translate into the desired amino acids.

Another means of increasing the number of carbohydrate moieties on the polypeptide is by chemical or enzymatic coupling of glycosides to the polypeptide. Such methods are described in the art, e.g., in WO 87/05330 published 11 Sep. 1987, and in Aplin and Wriston, CRC Crit. Rev. Biochem., pp. 259-306 (1981).

Removal of carbohydrate moieties present on the polypeptide may be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys., 259:52 (1987) and by Edge et al., Anal. Biochem., 118:131 (1981). Enzymatic cleavage of carbohydrate moieties on polypeptides can be achieved by the use of a variety of endo- and exo-glycosidases as described by Thotakura et al., Meth. Enzymol., 138:350 (1987).

Another type of covalent modification of cage moieties comprises linking the polypeptide to one of a variety of nonproteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192 or 4,179,337. This finds particular use in increasing the physiological half-life of the composition.

Cage polypeptides of the present invention may also be modified in a way to form chimeric molecules comprising an cage polypeptide fused to another heterologous polypeptide or amino acid sequence. In one embodiment, such a chimeric molecule comprises a fusion of a cage polypeptide with a tag polypeptide which provides an epitope to which an anti-tag antibody can selectively bind. The epitope tag is generally placed at the amino-or carboxyl-terminus of the polypeptide, although internal loops that are solvent exposed are also preferred. The presence of such epitope-tagged forms of a cage polypeptide can be detected using an antibody against the tag polypeptide. Also, provision of the epitope tag enables the cage polypeptide to be readily purified by affinity purification using an anti-tag antibody or another type of affinity matrix that binds to the epitope tag.

Various tag polypeptides and their respective antibodies are well known in the art. Examples include poly-histidine (poly-his) or poly-histidine-glycine (poly-his-gly) tags; the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology, 6:1204-1210 (1988)]; the KT3 epitope peptide [Martin et al., Science, 255:192-194 (1992)]; tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)].

In a preferred embodiment, the nanoparticles are derivatized for attachment to a variety of moieties, including but not limited to, dendrimer structures, additional proteins, carbohydrates, lipids, targeting moieities, etc. In general, one or more of the subunits is modified on an external surface to contain additional moieties.

In a preferred embodiment, a medical imaging agent is introduced into the protein cage. By “medical imaging agent” or “diagnostic agent” or “diagnostic imaging agent” herein is meant an agent that can be introduced into a cell, tissue, organ or patient and provide an image of the cell, tissue, organ or patient. Most methods of imaging make use of a contrast agent of one kind or another. Typically, a contrast agent is injected into the vascular system of the patient, and circulates through the body in, say, around half a minute. An image taken of the patient then shows enhanced features relating to the contrast agent. Diagnostic imaging agents include magnetic resonance imaging (MRI) agents, nuclear magnetic resonance (NMR) agents, x-ray imaging agents, optical imaging agents, ultrasound imaging agents and neutron capture therapy agents.

In a preferred embodiment, the medical imaging agent is a magnetic resonance imaging (MRI) agent. By “MRI agent” herein is meant a molecule that can be used to enhance the MRI image. MRI is a clinical diagnostic and research procedure that uses a high-strength magnet and radio-frequency signals to produce images. The most abundant molecular species in biological tissues is water. It is the quantum mechanical “spin” of the water proton nuclei that ultimately gives rise to the signal in imaging experiments. In MRI the sample to be imaged is placed in a strong static magnetic field (1-12 Tesla) and the spins are excited with a pulse of radio frequency (RF) radiation to produce a net magnetization in the sample. Various magnetic field gradients and other RF pulses then act on the spins to code spatial information into the recorded signals. MRI is able to generate structural information in three dimensions in relatively short time spans.

As is known in the art, MRI contrast agents generally comprise a paramagnetic metal ion bound to a chelator. By “paramagnetic metal ion”, “paramagnetic ion” or “metal ion” herein is meant a metal ion which is magnetized parallel or antiparallel to a magnetic field to an extent proportional to the field. Generally, these are metal ions which have unpaired electrons; this is a term understood in the art. Examples of suitable paramagnetic metal ions, include, but are not limited to, gadolinium III (Gd+3 or Gd(III)), iron III (Fe+3 or Fe(III)), manganese II (Mn+2 or Mn(II)), ytterbium III (Yb+3 or Yb(III)), dysprosium (Dy+3 or Dy(III)), and chromium (Cr(III) or Cr+3). In a preferred embodiment the paramagnetic ion is the lanthanide atom Gd(III), due to its high magnetic moment (u2=63BM2), a symmetric electronic ground state (S8), and its current approval for diagnostic use in humans.

In addition to the metal ion, the MRI contrast agent usually comprise a chelator. Due to the relatively high toxicity of many of the paramagnetic ions, the ions are rendered nontoxic in physiological systems by binding to a suitable chelator. The chelator utilizes a number of coordination atoms at coordination sites to bind the metal ion. There are a large number of known macrocyclic chelators or ligands which are used to chelate lanthanide and paramagnetic ions. See for example, Alexander, Chem. Rev. 95:273-342 (1995) and Jackels, Pharm. Med. Imag, Section III, Chap. 20, p645 (1990), expressly incorporated herein by reference, which describes a large number of macrocyclic chelators and their synthesis. Similarly, there are a number of patents which describe suitable chelators for use in the invention, including U.S. Pat. Nos. 5,155,215, 5,087,440, 5,219,553, 5,188,816, 4,885,363, 5,358,704, 5,262,532, and Meyer et al., Invest. Radiol. 25: S53 (1990), all of which are also expressly incorporated by reference. There are a variety of factors which influence the choice and stability of the chelate metal ion complex, including enthalpy and entropy effects (e.g. number, charge and basicity of coordinating groups, ligand field and conformational effects, etc.). In general, the chelator has a number of coordination atoms which are capable of binding the metal ion. The number of coordination atoms, and thus the structure of the chelator, depends on the metal ion. Thus, as will be understood by those in the art, any of the known paramagnetic metal ion chelators or lanthanide chelators can be easily modified using the teachings herein to add a functional moiety for covalent attachment to an optical dye or linker.

Preferred MRI contrast agents include, but are not limited to, 1,4,7,10-tetraazacyclododecane-N,N′,N″,N″′-tetracetic acid (DOTA), diethylenetriaminepentaacetic (DTPA), 1,4,7,10-tetraazacyclododecane-N,N′,N″,N″′-tetraethylphosphorus (DOTEP), 1,4,7,10-tetraazacyclododecane-N,N′,N″-triacetic acid (DOTA) and derivatives thereof (see U.S. Pat. Nos. 5,188,816, 5,358,704, 4,885,363, and 5,219,553, hereby expressly incorporated by reference).

In addition, as is known in the art, the use of iron oxides and aggregates of iron oxides as MRI agents is well known.

In a preferred embodiment, the medical imaging agent is a nuclear magnetic resonance imaging agent (NMR). By “NMR agent” herein is meant a molecule that can be used to enhance the NMR image. NMR is a very extensively used method of medical diagnosis, used for in vivo imaging, with which vessels of the body and body tissue (including tumors) can be visualized by measuring the magnetic properties of the protons in the body water. To this end, e.g., contrast media are used that produce contrast enhancement in the resulting images or make these images readable by influencing specific NMR parameters of the body protons (e.g., relaxation times T¹ and T²). Mainly complexes of paramagnetic ions, such as, e.g., gadolinium-containing complexes (e.g., Magnevist™) are used owing to the effect of the paramagnetic ions on the shortening of the relaxation times. A measure of the shortening of the relaxation time is relaxivity, which is indicated in m⁻¹ sec⁻¹.

For use in NMR imaging, paramagnetic ions (see above) are generally complexed with aminopolycarboxylic acids, e.g., with diethylenetriamine-pentaacetic acid [DTPA]). The di-N-methylglucamine salt of the Gd-DTPA complex is known under the name Magnevist™ and is used to diagnose tumors in the human brain and in the kidney. See U.S. Pat. No. 6,468,502 and EP 0 071 564 A1, both of which are incorporated by reference in their entirety.

In a preferred embodiment, the medical imaging agent is a x-ray agent. By “x-ray agent” herein is meant a molecule that can be used to enhance an x-ray image. Agents suitable for use as x-ray agents include contrast agents such as iodine or other suitable radioactive isotopes. See U.S. Pat. No. 6,219,572, incorporated by reference in its entirety.

In a preferred embodiment, the medical imaging agent is a optical agent. By “optical agent” herein is meant an agent comprising an “optical dye”. Optical dyes are compounds that will emit detectable energy after excitation with light. Optical dyes may be photoluminescent or fluorescent compounds. In a preferred embodiment, the optical dye is fluorescent; that is, upon excitation with a particular wavelength, the optical dye with emit light of a different wavelength; such light is typically unpolarized. In an alternative embodiment, the optical dye is phosphorescent.

Preferred optical dyes include, but are not limited to, fluorescein, rhodamine, tetramethyirhodamine, eosin, erythrosin, coumarin, methyl-coumarins, pyrene, Malacite green, stilbene, Lucifer Yellow, Cascade Blue™, and Texas Red. Suitable optical dyes are described in the 1989-1991 Molecular Probes Handbook by Richard P. Haugland, hereby expressly incorporated by reference.

In a preferred embodiment, the optical dye is functionalized to facilitate covalent attachment. Thus, a wide variety of optical dyes are commercially available which contain functional groups, including, but not limited to, isothiocyanate groups, amino groups, haloacetyl groups, maleimides, succinimidyl esters, and sulfonyl halides, all of which may be used to covalently attach the optical dye to a second molecule, such as other imaging agents or to the protein cages.

In a preferred embodiment, the medical imaging agent is a ultrasound agent. By “ultrasound agent” herein is meant an agent that can be used to generate an ultrasound image. Generally, for ultrasound, air in small bubble-like cells, i.e. microparticles is used as a contrast agent. See U.S. Pat. Nos. 6,219,572, 6,193,951, 6,165,442, 6,046,777, 6,177,062, all of which are hereby expressly incorporated by reference.

In a preferred embodiment, the medical imaging agent is a neutron capture therapy agent (NCT). NCT is based on the nuclear reaction produced when a neutron capture agent such as ¹⁰B or ¹⁵⁷Gd isotope (localized in tumor tissues) is irradiated with low energy thermal neutrons. The radiation produced is capable of effecting selective destruction of tumor cells while sparing normal cells. The advantage of NCT is the fact that it is a binary system, capable of independent variation of control of the neutron capture agent and thermal neutrons. Preferably, NCT agents comprise either ¹⁰B or ¹⁵⁷Gd. See U.S. Pat. Nos. 5,286,853, 6,248,305, and 6,086,837; all of which are hereby expressly incorporated by reference.

In a preferred embodiment, protein cages comprise a plurality of medical imaging agents.

The medical imaging agents may be the same or different. In a preferred embodiment, the medical imaging agents are the same.

In a preferred embodiment, the medical imaging agents are different. That is, a protein cage may comprise an MRI agent and an optical agent, or and MRI agent and an ultrasound agent, or an NCT and an optical agent, etc.

Regardless of whether the protein cage comprises the same or different imaging agents, anywhere from 1 to up to up 180 imaging agents may be entrapped within a protein cage. In some embodiments, the imaging agents may be attached to polymers (described below) and under these conditions, from 10 to 1000 imaging agents may be entrapped in a protein cage.

In a preferred embodiment, a therapeutic agent is introduced into the protein cage. By “therapeutic agent” or “drug moiety” or therapeutically active agent” herein is meant an agent must be capable of effecting a therapeutic effect, i.e. it alters a biological function of a physiological target substance. By “causing a therapeutic effect” or “therapeutically effective” or grammatical equivalents herein is meant that the drug alters the biological function of its intended physiological target in a manner sufficient to cause a therapeutic and phenotypic effect. By “alters” or “modulates the biological function” herein is meant that the physiological target undergoes a change in either the quality or quantity of its biological activity; this includes increases or decreases in activity. Thus, therapeutically active agents include a wide variety of drugs, including antagonists, for example enzyme inhibitors, and agonists, for example a transcription factor which results in an increase in the expression of a desirable gene product (although as will be appreciated by those in the art, antagonistic transcription factors may also be used), are all included.

The nature of the therapeutic effect between the therapeutically active moiety and the physiological target substance will depend on the both the physiological target substance and the nature of the effect. In general, suitable physiological target substances include, but are not limited to, proteins (including peptides and oligopeptides) including ion channels and enzymes; nucleic acids; ions such as Ca+2, Mg+2, Zn+2, K+, Cl−, Na+, and toxic ions including those of Fe, Pb, Hg and Se; cAMP; receptors including G-protein coupled receptors and cell-surface receptors and ligands; hormones; antigens; antibodies; ATP; NADH; NADPH; FADH2; FNNH2; coenzyme A (acyl CoA and acetyl CoA); and biotin, among others. Physiological target substances include enzymes and proteins associated with a wide variety of viruses including orthomyxoviruses, (e.g. influenza virus), paramyxoviruses (e.g respiratory syncytial virus, mumps virus, measles virus), adenoviruses, rhinoviruses, coronaviruses, reoviruses, togaviruses (e.g. rubella virus), parvoviruses, poxviruses (e.g. variola virus, vaccinia virus), enteroviruses (e.g. poliovirus, coxsackievirus), hepatitis viruses (including A, B and C), herpesviruses (e.g. Herpes simplex virus, varicella-zoster virus, cytomegalovirus, Epstein-Barr virus), rotaviruses, Norwalk viruses, hantavirus, arenavirus, rhabdovirus (e.g. rabies virus), retroviruses (including HIV, HTLV-I and -II), papovaviruses (e.g. papillomavirus), polyomaviruses, and picornaviruses, and the like. Similarly, bacterial targets can come from a wide variety of pathogenic and non-pathogenic prokaryotes of interest including Bacillus; Vibrio, e.g. V. cholerae; Escherichia, e.g. Enterotoxigenic E. coli, Shigella, e.g. S. dysenteriae; Salmonella, e.g. S. typhi; Mycobacterium e.g. M. tuberculosis, M. leprae; Clostridium, e.g. C. botulinum, C. tetani, C. difficile, C.perfringens; Cornyebacterium, e.g. C. diphtheriae; Streptococcus, S. pyogenes, S. pneumoniae; Staphylococcus, e.g. S. aureus; Haemophilus, e.g. H. influenzae; Neisseria, e.g. N. meningitidis, N. gonorrhoeae; Yersinia, e.g. Y. pestis, Pseudomonas, e.g. P. aeruginosa, P. putida; Chlamydia, e.g. C. trachomatis; Bordetella, e.g. B. pertussis. Finally other targets can include Treponema, e.g. T. palladium; G. lamblia and the like.

Once the physiological target substance has been identified, a corresponding therapeutically active agent is chosen. These agents will be any of a wide variety of drugs, including, but not limited to, enzyme inhibitors, hormones, cytokines, growth factors, receptor ligands, antibodies, antigens, ion binding compounds including crown ethers and other chelators, substantially complementary nucleic acids, nucleic acid binding proteins including transcription factors, toxins, etc. Suitable drugs include cytokines such as erythropoietin (EPO), thrombopoietin (TPO), the interleukins (including IL-1 through IL-17), insulin, insulin-like growth factors (including IGF-1 and -2), epidermal growth factor (EGF), transforming growth factors (including TGF-α and TGF-β), human growth hormone, transferrin, epidermal growth factor (EGF), low density lipoprotein, high density lipoprotein, leptin, VEGF, PDGF, ciliary neurotrophic factor, prolactin, adrenocorticotropic hormone (ACTH), calcitonin, human chorionic gonadotropin, cotrisol, estradiol, follicle stimulating hormone (FSH), thyroid-stimulating hormone (TSH), leutinzing hormone (LH), progeterone, testosterone, toxins including ricin, and any drugs as outlined in the Physician's Desk Reference, Medical Economics Data Production Company, Montvale, N.J., 1998 and the Merck Index, 11th Edition (especially pages Ther-1 to Ther-29), both of which are expressly incorporated by reference.

In a preferred embodiment, the therapeutically active compound is a drug used to treat cancer. Suitable cancer drugs include, but are not limited to, antineoplastic drugs, including alkylating agents such as alkyl sulfonates (busulfan, improsulfan, piposulfan); aziridines (benzodepa, carboquone, meturedepa, uredepa); ethylenimines and methylmelamines (altretamine, triethylenemelamine, triethylenephosphoramide, triethylenethiophosphoramide, trimethylolmelamine); nitrogen mustards (chlorambucil, chlomaphazine, cyclophosphamide, estramustine, ifosfamide, mechlorethamine, mechlorethamine oxide hydrochloride, melphalan, novembichin, phenesterine, prednimustine, trofosfamide, uracil mustard); nitrosoureas (carmustine, chlorozotocin, fotenmustine, lomustine, nimustine, ranimustine); dacarbazine, mannomustine, mitobranitol, mitolactol; pipobroman; doxorubicin, and cisplatin (including derivatives).

In a preferred embodiment, the therapeutically active compound is a peptide used to treat cancer. Preferably, the peptide is laminin peptide 11 (see above).

In a preferred embodiment, the therapeutically active compound is an antiviral or antibacterial drug, including aclacinomycins, actinomycin, anthramycin, azaserine, bleomycins, cuctinomycin, carubicin, carzinophilin, chromomycins, ductinomycin, daunorubicin, 6-diazo-5-oxn-1-norieucine, duxorubicin, epirubicin, mitomycins, mycophenolic acid, nogalumycin, olivomycins, peplomycin, plicamycin, porfiromycin, puromycin, streptonigrin, streptozocin, tubercidin, ubenimex, zinostatin, zorubicin; aminoglycosides and polyene and macrolide antibiotics.

In a preferred embodiment, the therapeutically active compound is a radio-sensitizer drug.

In a preferred embodiment, the therapeutically active compound is an anti-inflammatory drug (either steroidal or non-steroidal).

In a preferred embodiment, the therapeutically active compound is involved in angiogenesis. Suitable moieties include, but are not limited to, endostatin, angiostatin, interferons, platelet factor 4 (PF4), thrombospondin, transforming growth factor beta, tissue inhibitors of metalloproteinase -1, -2 and -3 (TIMP-1, -2 and -3), TNP-470, Marimastat, Neovastat, BMS-275291, COL-3, AG3340, Thalidomide, Squalamine, Combrestastatin, SU5416, SU6668, IFN-α, EMD121974, CAI, IL-12 abnd IM862.

In a preferred embodiment, the physiological target is a protein that contains a histidine residue that is important for the protein's bioactivity. In this case, the therapeutically active agent can be a metal ion complex (not to be confused with the metal ion complexes of the imaging agents), such as is generally described in PCT US95/16377, PCT US95/16377, PCT US96/19900, PCT US96/15527, and references cited within, all of which are expressly incorporated by reference. These cobalt complexes have been shown to be efficacious in decreasing the bioactivity of proteins, particularly enzymes, with a biologically important histidine residue. These cobalt complexes appear to derive their biological activity by the substitution or addition of ligands in the axial positions. The biological activity of these compounds results from the binding of a new axial ligand, most preferably the nitrogen atom of imidazole of the side chain of histidine which is required by the target protein for its biological activity. Thus, proteins such as enzymes that utilize a histidine in the active site, or proteins that use histidine, for example, to bind essential metal ions, can be inactivated by the binding of the histidine in an axial ligand position of the cobalt compound, thus preventing the histidine from participating in its normal biological function.

In a preferred embodiment, the physiological target protein is an enzyme. As will be appreciated by those skilled in the art, the possible enzyme target substances are quite broad. Suitable classes of enzymes include, but are not limited to, hydrolases such as proteases, carbohydrases, lipases and nucleases; isomerases such as racemases, epimerases, tautomerases, or mutases; transferases, kinases and phophatases. Enzymes associated with the generation or maintenance of arterioschlerotic plaques and lesions within the circulatory system, inflammation, wounds, immune response, tumors, apoptosis, exocytosis, etc. may all be treated using the present invention. Enzymes such as lactase, maltase, sucrase or invertase, cellulase, α-amylase, aldolases, glycogen phosphorylase, kinases such as hexokinase, proteases such as serine, cysteine, aspartyl and metalloproteases may also be detected, including, but not limited to, trypsin, chymotrypsin, and other therapeutically relevant serine proteases such as tPA and the other proteases of the thrombolytic cascade; cysteine proteases including: the cathepsins, including cathepsin B, L, S, H, J, N and O; and calpain; and caspases, such as caspase-3, -5, -8 and other caspases of the apoptotic pathway, such as interleukin-converting enzyme (ICE). Similarly, bacterial and viral infections may be detected via characteristic bacterial and viral enzymes. As will be appreciated in the art, this list is not meant to be limiting.

Once the target enzyme is identified or chosen, enzyme inhibitor therapeutically active agents can be designed using well known parameters of enzyme substrate specificities. As outlined above, the inhibitor may be another metal ion complex such as the cobalt complexes described above. Other suitable enzyme inhibitors include, but are not limited to, the cysteine protease inhibitors described in PCT US95/02252, PCT/US96/03844 and PCT/US96/08559, and known protease inhibitors that are used as drugs such as inhibitors of HIV proteases.

In one embodiment, the therapeutically active agent is a nucleic acid, for example to do gene therapy or antisense therapy. By “nucleic acid” or “oligonucleotide” or grammatical equivalents herein means at least two nucleotides covalently linked together. A nucleic acid of the present invention will generally contain phosphodiester bonds, although in some cases, as outlined below, nucleic acid analogs are included that may have alternate backbones, comprising, for example, phosphoramide (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev. (1995) pp 169-176). Several nucleic acid analogs are described in Rawls, C & E News Jun. 2, 1997 page 35. All of these references are hereby expressly incorporated by reference. These modifications of the ribose-phosphate backbone may be done to increase the stability and half-life of such molecules in physiological environments; for example, PNA antisense embodiments are particularly preferred.

As will be appreciated by those in the art, all of these nucleic acid analogs may find use in the present invention. In addition, mixtures of naturally occurring nucleic acids and analogs can be made or mixtures of different nucleic acid analogs.

The nucleic acid may be single-stranded or double stranded. The physiological target molecule can be a substantially complementary nucleic acid or a nucleic acid binding moiety, such as a protein.

In a preferred embodiment, the physiological target substance is a physiologically active ion, and the therapeutically active agent is an ion binding ligand or chelate. For example, toxic metal ions could be chelated to decrease toxicity, using a wide variety of known chelators including, for example, crown ethers.

In some embodiments, therapeutic agent and targeting moiety can be the same. In a preferred embodiment, laminin peptide 11 is used as both a targeting moiety and a therapeutic agent.

In a preferred embodiment, the invention provides delivery agents comprising catalytic centers. That is, either in addition to or instead of the imaging agents of the invention, the cages of the invention include a catalytic center that delivers an activity to the cell or tissue that is then used to generate a desirable result. For example, enzymes, including enzyme mimics, can be delivered in this way. One example of an enzyme mimic is a complex of copper bound to phenanthroline, which acts as a non-specific hydrolase of nucleic acids; thus it may be used to hydrolyze exogeneous nucleic acid in a cell, for example in the case of viral infection (see Sigman, D. S. 1986, Acc. Chem. Res. 19:180-186;and Davies, R. R. and Distefano, M.D., 1997, J. Am. Chem. Soc. 119:11643-11652). Similarly, any of the enzymatic activities outlined above can be delivered as well, for any number of purposes. Furthermore, metal-based catalysts are used in a wide variety of contexts that can be included in the delivery agents of the invention, for example to turn prodrugs into drugs.

In addition to the components outlined above, it should be appreciated that the imaging agents and therapeutic agents of the present invention may be attached to the protein cage via a linker. Linkers are well known in the art; for example, homo-or hetero-bifunctional linkers as are Well known (see 1994 Pierce Chemical Company catalog, technical section on cross-linkers, pages 155-200, incorporated herein by reference). Generally, suitable linker groups include, but are not limited to, alkyl and aryl groups, including substituted alkyl and aryl groups and heteroalkyl (particularly oxo groups) and heteroaryl groups, including alkyl amine groups, as defined above. Preferred linker groups include p-aminobenzyl, substituted p-aminobenzyl, diphenyl and substituted diphenyl, alkyl furan such as benzylfuran, carboxy, and straight chain alkyl groups of 1 to 10 carbons in length, short alkyl groups, esters, amide, amine, epoxy groups, nucleic acids, peptides and ethylene glycol and derivatives. Particularly preferred linkers include p-aminobenzyl, methyl, ethyl, propyl, butyl, pentyl, hexyl, acetic acid, propionic acid, aminobutyl, p-alkyl phenols, 4-alkylimidazole and polymers. The selection of the linker group is generally done using well known molecular modeling techniques, to optimize the obstruction of the coordination site or sites of the metal ion. In addition, the length of this linker may be very important in order to achieve optimal results.

In a preferred embodiment, the linker used to attach the imaging agent and therapeutic agents to a protein cage is a polymer. As will be appreciated by those of skill in the art, polymers comprising only imaging agents, only therapeutic agents or a combination of both have use in the methods of the invention. Moreover, protein cages comprising imaging agents and therapeutic agents may also be attached to polymers via functional groups introduced on the surface of the cage (see above).

The character of the polymer will vary, but what is important is that the polymer either contain or can be modified to contain functional groups for the attachment of the nanoparticles of the invention. Suitable polymers include, but are not limited to, functionalized dextrans, styrene polymers, polyethylene and derivatives, polyanions including, but not limited to, polymers of heparin, polygalacturonic acid, mucin, nucleic acids and their analogs including those with modified ribose-phosphate backbones, the polypeptides polyglutamate and polyaspartate, as well as carboxylic acid, phosphoric acid, and sulfonic acid derivatives of synthetic polymers; and polycations, including but not limited to, synthetic polycations based on acrylamide and 2-acrylamido-2-methylpropanetrimethylamine, poly(N-ethyl-4-vinylpyridine) or similar quarternized polypyridine, diethylaminoethyl polymers and dextran conjugates, polymyxin B sulfate, lipopolyamines, poly(allylamines) such as the strong polycation poly(dimethyldiallylammonium chloride), polyethyleneimine, polybrene, spermine, spermidine and polypeptides such as protamine, the histone polypeptides, polylysine, polyarginine and polyornithine; and mixtures and derivatives of these. Particularly preferred polycations are polylysine and spermidine. Both optical isomers of polylysine can be used. The D isomer has the advantage of having long-term resistance to cellular proteases. The L isomer has the advantage of being more rapidly cleared from an animal when administered. As will be appreciated by those in the art, linear and branched polymers may be used.

A preferred polymer is polylysine, as the —NH2 groups of the lysine side chains at high pH serve as strong nucleophiles for multiple attachment of imaging and therapeutic agents. At high pH the lysine monomers can be coupled to the nanoparticles under conditions that yield on average 5-20% monomer substitution.

The size of the polymer may vary substantially. For example, it is known that some nucleic acid vectors can deliver genes up to 100 kilobases in length, and artificial chromosomes (megabases) have been delivered to yeast. Therefore, there is no general size limit to the polymer. However, a preferred size for the polymer is from about 10 to about 50,000 monomer units, with from about 2000 to about 5000 being particularly preferred, and from about 3 to about 25 being especially preferred.

In general, the protein cages are made recombinantly and self assemble upon contact (or by alteration of their chemical environment; see Examples). As will be appreciated by those in the art, there are a wide variety of available techniques for the production of proteins in a wide variety of organisms.

In a preferred embodiment, a yeast-based heterologous protein expression system (Pichia pastoris) for the large scale production of modified CCMV protein cages (see Example 5) Alternatively, an E. coli—based CCMV coat protein expression system can be used (Zhao, X., et al., 1995. Virology 207:486-494). Using the E. coli system, denatured coat protein can be purified to 90% homogeneity, renatured, and assembled into empty particles which are indistinguishable from native particles (Fox, J. M., et al., 1998, Virology 244:212-218; and Zhao, X., et al., 1995, Virology 207:486-494).

Once made, the protein cages are loaded with medical imaging agents and/or therapeutic agents. By “loaded” or “loading” or grammatical equivalents herein is meant the introduction of imaging agents, therapeutic agents and other non-native materials (sometimes referred to herein as “guest molecules”) into the interior of the protein shell (also referred to herein as “crystallization” or “mineralization” depending on the material loaded). As will be appreciated by those of skill in the art, loading includes the synthesis of materials within the shell.

In preferred embodiments, the protein shells are empty. By “empty” herein is meant that the cages are prepared lacking materials that would commonly be contained within. For example, if viral protein cages are used, the shells are prepared lacking viral nucleic acids and proteins.

In general, there are two ways to control the size of the core; by altering the cage size, as outlined herein, or by controlling the material to protein shell ratio (e.g. the loading factor). That is, by controlling the amount of available material as a function of the amount and size of shells to be loaded, the loading factor of each individual particle can be adjusted. For example, as outlined below, mammalian ferritin shells can generally accommodate as many as 4,000 iron atoms, while protein cages from Listeria can accommodate 500. These presumably maximum numbers may be decreased by decreasing the load factors. In general, the loading is an equilibrium driven passive event or entrapment (although as outlined below, the natural channels or “holes” in the shells can be manipulated to alter these parameters), with physiological buffers, temperature and pH being preferred, with loading times of 12-24 hours.

In a preferred embodiment, the protein cage is loaded via the use of a chemical switch. For example, if a pH sensitive switching mechanism is used, empty cages are dialyzed in a saturated solution comprising an imaging agent, or an imaging agent and a therapeutic agent at room temperature and at pH>6.5 to ensure that that the cage is in an open conformation. Once crystallization has been initiated, the pH of the solution is lowered, i.e. pH<6.5 to switch the cage to a closed formation. The resulting cage with its entrapped imaging agent, etc., is then isolated using gradient centrifugation or column chromatography. If desired, the cages can be isolated prior to bulk crystallization and counter ions, such as Me₄N⁺ added to induce crystal formation. Once isolated, the cages may be stored or used directly.

In alternative methods, the protein cage is loaded using a redox sensitive switch. In this embodiment, the redoxing conditions are altered. For example, reducing conditions should favor cage expansion, i.e., open conformation, via the breaking of disulfide bonds and entrapment of imaging agents. Alternatively, oxidizing conditions should prevent expansion and thereby the release of entrapped agents.

Once made, the compositions of the invention find use in a variety of applications. Preferably, the compositions are used in a variety of imaging and therapeutic applications. For example, once synthesized, the metal ion complexes of the invention have use as magnetic resonance imaging contrast or enhancement agents. Specifically, the imaging agents of the invention have several important uses, including the non-invasive imaging of drug delivery, imaging the interaction of the drug with its physiological target, monitoring gene therapy, in vivo gene expression (antisense), transfection, changes in intracellular messengers as a result of drug delivery, etc.

Delivery agents comprising imaging agents comprising metal ions may be used in a similar manner to the known gadolinium MRI agents. See for example, Meyer et al., supra; U.S. Pat. No. 5,155,215; U.S. Pat. No. 5,087,440; Margerstadt et al., Magn. Reson. Med. 3:808 (1986); Runge et al., Radiology 166:835 (1988); and Bousquet et al., Radiology 166:693 (1988). The metal ion complexes are administered to a cell, tissue or patient as is known in the art.

Delivery agents comprising imaging agents that do not use metal ions may be used in a similar manner as described in U.S. Pat. Nos. 6,219,572, 6,219,572, 6,193,951, 6,165,442, 6,046,777, 6,177,062, 5,286,853, 6,248,305, and 6,086,837, all of which are hereby expressing incorporated by reference.

A “patient” for the purposes of the present invention includes both humans and other animals and organisms, such as experimental animals. Thus the methods are applicable to both human therapy and veterinary applications. In addition, the metal ion complexes of the invention may be used to image tissues or cells; for example, see Aguayo et al., Nature 322:190 (1986).

Generally, sterile aqueous solutions of the imaging agent complexes of the invention are administered to a patient in a variety of ways, including orally, intrathecally and especially intraveneously in concentrations of 0.003 to 1.0 molar, with dosages from 0.03, 0.05, 0.1, 0.2, and 0.3 millimoles per kilogram of body weight being preferred. Dosages may depend on the structures to be imaged. Suitable dosage levels for similar complexes are outlined in U.S. Pat. Nos. 4,885,363 and 5,358,704.

In a preferred embodiment, the compositions of the invention are used to deliver therapeutic agents to patients. The administration of the compositions of the present invention can be done in a variety of ways, including, but not limited to, orally, subcutaneously, intravenously, intranasally, transdermally, intraperitoneally, intramuscularly, intrapulmonary, vaginally, rectally, or intraocularly. In some instances, for example, in the treatment of wounds and inflammation, the composition may be directly applied as a solution or spray. Depending upon the manner of introduction, the cages may be formulated in a variety of ways, including as polymers, etc. The concentration of therapeutically active compound in the formulation may vary from about 0.1-100 wt. %.

In a preferred embodiment, the compositions of the invention further comprise therapeutic agents for administration to patients. The administration of the compositions of the present invention can be done in a variety of ways, including, but not limited to, orally, subcutaneously, intravenously, intranasally, transdermally, intraperitoneally, intramuscularly, intrapulmonary, vaginally, rectally, or intraocularly in concentrations of 0.003 to 1.0 molar, with dosages from 0.03, 0.05, 0.1, 0.2, and 0.3 millimoles per kilogram of body weight being preferred. In some instances, for example, in the treatment of wounds and inflammation, the composition may be directly applied as a solution or spray. Depending upon the manner of introduction, the compositions may be formulated in a variety of ways, including as polymers, etc. The concentration of therapeutically active compound in the formulation may vary from about 0.1-100 wt. %.

Generally, pharmaceutical compositions for use with both imaging and therapeutic agents are in a water soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

The pharmaceutical compositions may also include one or more of the following: carrier proteins such as serum albumin; buffers; fillers such as microcrystalline cellulose, lactose, corn and other starches; binding agents; sweeteners and other flavoring agents; coloring agents; and polyethylene glycol. Additives are well known in the art, and are used in a variety of formulations.

Combinations of the compositions may be administered. Moreover, the compositions may be administered in combination with other therapeutics.

In some embodiments, it may be desirable to increase the blood clearance times (or half-life) of the delivery agent compositions of the invention. This has been done, for example, by adding carbohydrate polymers, including polyethylene glycol, to other compositions as is known in the art.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. All references cited herein, including U.S. Ser. No. 60/380,942, are incorporated by reference in their entirety.

### EXAMPLES

### Example 1

### Modifications to Protein Cages for Enhanced Gd³⁺ Binding

We have taken advantage of our structural knowledge of the Ca²⁺ binding in wild type virions in an attempt to enhance binding of gadolinium (Gd³⁺) for eventual use as a possible MRI contrast agent. The Ca²⁺ binding sites in wild type virions results from the precise orientation of acidic residues contributed from adjacent coat protein subunits at the quasi three-fold axis (Speir, J. A., et al., 1995, Structure 3:63-78; and Zhao, X., 1998, Ph.D. Purdue University). There are 180 Ca²⁺ binding sites per virion. Ca²⁺ binding at these sites is thought to satisfy the charge repulsion created at pH 6.5 by the cluster of acidic residues, and to assist with creating shell curvature during virion assembly. Ca²⁺ is normally required for in vitro assembly of CCMV at >pH 6.5. We have demonstrated that Gd³⁺ can act as a substitute for Ca²⁺ in the pH-dependent assembly assay. We are attempting to enhance assembly-dependent Gd³⁺ binding by protein engineering of the Ca²⁺ site. The ionic radii of Ca²⁺ and Gd³⁺ are quite similar (0.99 and 0.938 Å respectively) indicating that the Ca²⁺ binding site is already a good starting point for Gd³⁺ binding. In general the Lanthanides prefer O and N donor atoms in their coordination environment and show considerable variability in coordination number. Therefore, we have introduced a combination of glutamic acid and histidine residues at positions 86 (Q86E/H) and/or 149 (Q149E/H) which are in close proximity to the metal and would provide one or two additional coordination sites for the metal. These constructs have been confirmed by DNA sequencing and are in the process of being expressed in the P. pastoris system.

Genetic Modifications to the Ca²⁺ Binding Sites

A collection of coat protein mutations surrounding the CCMV Ca²⁺ binding site (Glu81, Gln85, Glu148, Gln 149, Asp 153) will be produced and assayed for their ability to create high affinity Gd³⁺ binding. All of the Gd³⁺ binding mutations will be made in a R26C/K42R mutant coat protein background that allows for the highly stable assembly of empty particles in the P. pastoris expression system. We propose to add to the R26C/K42R coat protein background a combination of Q85H/E and Q149H/E mutations to facilitate enhanced Gd³⁺ binding. The rationale for this series of mutations is that changing the glutamine positions in the Ca^{2 +} binding site to either an acidic glutamic acid or histidine will provide additional coordinating ligands for enhanced binding of Gd³⁺. These mutations will be created as a single mutation and in double mutation combinations using PCR oligonucleotide site-directed mutagenisis protocols that are well established in our laboratories. A total of 8 mutations are to be generated: Q85H, Q85E, Q149H, Q149E, Q85H:Q149H, Q85H:Q149E, Q85E:Q149H, Q85E:Q149E. These modifications involve minimal changes in the coordination geometry of the metal (all modified residues do coordinate the metal in the endogenous site) but rather involve the change from weak electron donor (Gln) to strong electron donor (Glu or His) capable of binding the Gd³⁺. We will confirm each of the mutations by DNA sequencing and by the use of a coupled in vitro translation/transcription assays that determines that each mutation/clone is capable of producing the full-length coat protein. Once confirmed, the clones will be introduced into the P. pastoris expression system by methods well established in our laboratories. The mutated form of empty particles will be purified to near homogeneity on sucrose gradients as previously described. If some or all of the Gd³⁺ mutations fail to assemble into empty particles, the expressed coat protein will be purified by Ni²⁺ affinity chromatography using a poly-histidine tag present on the N-terminus of the coat protein. We have previously demonstrated that the R26C/K42R coat protein containing the poly histidine tag can be purified from P. pastoris to near homogeneity by Ni-affinity chromatography. The purified protein is efficiently assembled in vitro into empty virus particles.

Quantifying Gd³⁺ Binding

The ability of the engineered CCMV virion to bind Gd³⁺ will be assessed by in vitro assembly, fluorescence quenching, and isothermal titration calorimetry. Both fluorescence quenching and isothermal titration calorimetry will allow us to determine the binding constant for Gd to the engineered virus as compared to control experiments using the wild type virion and mutants with disrupted metal binding sites. These techniques provide two very sensitive and complementary methods for measuring the binding of Gd³⁺ to the virion—an essential component for developing this technology for medical diagnostic purposes.

In vitro Assembly Assay

The in vitro assembly assay takes advantage of the fact that particle formation is dependent on Gd³⁺ at values >pH 6.5 (wild-type coat protein is dependent on Ca²⁺). After purification (either as assembled empty cages or as non-assembled protein) the eight Gd^{3 +} binding mutants will be disassembled under high ionic strength, elevated pH and temperature, and a reducing environment. (0.5M CaCl₂, 5 mM DTT, pH 8.5 at 50° C.). The disassembled coat protein will be further purified by Sephadex G-100 size exclusion chromatography (the coat protein usually exists in its non-covalent dimer form). All residual Ca²⁺ is removed by extensive dialysis with EGTA. Gd³⁺-dependent assembly of the protein into particles is assayed by dialyzing 0.1 mg/ml (100 μl) of the coat protein-Gd³⁺ binding mutations in the presence of increasing amounts of Gd³⁺ (0-1 mM Gd³⁺) at pH 7.0, 20° C. The amount of assembly is determined by sedimentation on 10-40% sucrose gradients and quantitating the amount of the free coat protein (3S) vs. assembled empty particles (50S). A second quantitative ELISA assay will also be used that only detects assembled virions and not the non-assembled coat protein.

Fluorescence Quenching

The endogenous metal binding site in CCMV is roughly 1.4 nm from Trp55 and 1.5 nm from Trp47. The fluorescence behavior of the Trp55 and 47 is expected to be quenched by energy transfer to the metal ion occupying the site close to it as has been demonstrated in related metal binding proteins (Treffry, A., et al., 1998, J. Biol. Inorg Chem. 3:682-688). Excitation of the protein at 280 nm leads to fluorescence emission from Trp at 340 nm. The assembled metal free virion (1 mg in 1.0 ml 0.1 M MES, pH 6.5) will be titrated by small additions (2 μL) of Gd³⁺ (20 mM) and the fluorescence will be measured at 340 nm after excitation at 280 nm. The Gd additions will be continued until the metal binding sites are saturated and no further decrease in fluorescence can be detected. In this way we can monitor the steady state fluorescence quenching as a function of [Gd³⁺] concentration. This is expected to give a simple hyperbolic ligand binding curve which can be fit to extract the dissociation (or binding) constant K_{d}. Controls for this measurement will include the wild type virion and CCMV expressing E81T/E85T and E149T modifications which we have shown previously eliminates Ca²⁺ binding. This technique requires only modest amounts of protein which can be easily recovered from the experiment.

Isothermal Titration Microcalorimetry

Isothermal titration microcalorimetry allows us to measure directly the heat evolved as two or more species interact (Wadsö, I., 1997, Chem. Soc. Rev. 26:79-86.). Thus, a solution of the assembled, metal-free virion can be titrated with a solution of Gd³⁺. The empty virion from P. pastoris will be isolated as described previously and then dialyzed extensively against chelating agent (EDTA/EGTA) in low pH buffer (0.1 M Ac pH 4.5) to ensure metal removal whilst maintaining virion assembly. Excess chelating agent will then be removed by further dialysis against buffer. Immediately prior to calorimetry experiments, the virion will be re-purified by gel filtration (with 10⁶ M_{w} cut-off) to ensure that only fully assembled virions will be assayed. A solution of the virion (2 ml of 0.5-1 mg/ml) will be titrated with Gd(III) (20 mM) using 5.0 μL injections and allowing 8 minutes between injections for baseline re-equilibration for a total of 20 injections. The heat evolved during the reaction is monitored by heat compensation using a MicroCal titration calorimeter and recorded. The curve can then be fit (using Origin) to accommodate a binding model, and a binding constant (or dissociation constant) can be extracted. By measuring the heat directly this technique allows simultaneous determination of all binding parameters (K, ΔH°, ΔS°, and n the number of sites) in a single experiment (Wiseman, T., et al., 1989, Analytical Biochemistry 179:131-137.). In the case of the CCMV virion we expect 180 equivalent Gd³⁺ binding sites.

Evaluation of Gd-bound CCMV Virion as a Potential Candidate for MRI Contrast Agent

Empty virions (those with and without the Pep-11 fusion protein) will be purified from P. pastoris as previously described. Endogenous Ca²⁺ will be removed from the virion by extensive chelation with EDTA/EGTA and the metal-free virion loaded with Gd(III). Gd loaded virions will be isolated by either gel filtration or gradient centrifugation. The virions will initially be characterized by their effect on the values for T1 relaxation of water protons, which will be determined by ¹H NMR. This will require approximately 0.5 ml of 2 mg/ml Gd loaded virion. T1 will be measured by inversion recovery experiment (180°ₓ-θ-90°ₓ-FID) on a GE 400 MHz NMR. However, this T1 is measured at a single field strength and it is of particular interest for the development of these materials as MRI contrast agents to measure T1 (and T2) as a function of field strength.

Animal Studies and Scintigraphic Imaging

A limited number of animal studies are proposed for the express purpose of determining the biodistribution and blood half life of the virions. We have chosen to perform these initial experiments with ^{99m}Tc because the Tc radioactive assay is a very sensitive technique that is preferred for determining the biodistribution of wild-type and pep-11 modified virions in a mouse model system. Synthesis and attachment of a specific ligand for Tc (nicotinyl hydrazine (2)) to exposed lysine residues on the wild-type virion has already been achieved. Initial ^{99m}Tc radioisotope labeling studies have shown that we can incorporate significant amount of ^{99m}Tc into the nicotinyl hydrazine modified virion of CCMV.

After modification and radiolabeling with ^{99m}Tc, CCMV protein cages (both wild-type and those showing good cell binding activity and competition with free peptide 11 in vitro) will be tested and imaged in mice. The animals will each be given a single injection of radiolabeled protein cages via the tail vein, then returned to their cages. Three different radiolabeled forms will be tested on groups of mice: ^{99m}Tc -protein cages consisting of wild-type virions, ^{99m}Tc -protein cages which have been modified with Pep-11, and ^{99m}Tc -labeled free peptide 11. The radiolabeling of these forms will be carried out as described above using the well established nicotinyl hydrazine ligand (Abrams, M. J., et al., 1990, J. Nucl. Med. 31:2022-2028.). At various times after dosing, groups of animals will be euthanized with an overdose of halothane in a chamber. Each animal will be placed on the surface of the gamma camera for collection of a scintigraphic image of the distribution of radioactivity in its body. The camera will be fitted with a low-energy all-purpose collimator and the energy discriminator of the camera will be set to acquire the 140 keV photon of Tc-99m with a 20% window. The images will be captured in digital form by a NucLear Mac computer which is interfaced to the gamma camera. Immediately afterward, the major organs and tissues will be removed, weighed and counted in a scintillation counter to assess radiotracer content. Organs and tissues to be collected will include: liver, spleen, lungs, kidneys, stomach, small and large intestine, bone, muscle, and blood. The amount of radioactivity in each tissue will be expressed as a percent of the injected dose, determined from an appropriately diluted standard of the initial radiotracer obtained before injection.

### Example 2

### Electrostatic Modifications to Protein Cages

Entrapment and Growth of Anionic Metal Species

We have crystallized a range of polyoxometalate species in CCMV and the Norwalk Virus. This was accomplished by providing an interface for molecular aggregation, based on complementary electrostatic interactions between the protein and the anion metal species, which creates a locally high concentration at the protein interface. Briefly outlined, the empty virions were incubated with the precursor ions (WO₄²⁻, VO₃⁻, MoO₄²⁻) at approximately neutral pH. Under these conditions the virus exists in its open (swollen) form and allows all ions access to the cavity. The pH of the virus solution was then lowered to approximately pH 5.0. This induced two important complementary effects i) The inorganic species underwent a pH dependent oligomerization to form large polyoxometalate species such as H₂WO₄₂¹⁰⁻ (Douglas, T., and M. J. Young., 1998, Nature 393:152-155) which were readily crystallized as ammonium salts ii) the viral capsid particle underwent a structural transition in which the pores in the protein shell closed, trapping crystallized mineral or mineral nuclei within the virus. Crystal growth of the polyoxometallate salt continued until the virion container was filled. Thus, the material synthesized is both size and shape constrained by the size and shape of the interior of the viral protein cage. The resulting product(s) could be easily purified (by sedimentation velocity centrifugation on sucrose gradients, density centrifugation on cesium gradients or size exclusion chromatography), as it maintained all the same physical characteristics of the virion itself, and was visualized by transmission electron microscopy (FIGS. 6A and 6B). Experimental conditions were adjusted so that mineralization occurred selectively only within the viral capsid and no bulk mineralization was observed in solutions containing assembled viral capsids or virion-free controls. We postulate that the role of the virion is to provide a highly charged interface capable of binding the polyoxometalate polyanions and thus providing stabilization of incipient crystal nuclei. The protein interface thus acts as a nucleation catalyst (Hulliger, J., 1994, Angew. Chem. Int. Ed. 33:143-162.) based on the electrostatic potential generated by Arg and Lys residues which constitute the native RNA binding sites.

Entrapment and Growth of Soft Metal Species

Recently, we have begun to investigate how changes in the electrostatic nature of the N-terminus affect the type of materials that can be entrapped within the virus particles. As an example, we have investigated the catalytic binding properties of a mutant form of CCMV which has a specific metal binding site (6 histidines) engineered onto the N-terminus of the coat protein. Histidine has very high affinity for so-called soft metals including Fe(II) (Cotton, F. A., and G. Wilkinson., 1999, Inorganic Chemistry, John Wiley & Sons). We have found that this mutant is able to selectively bind Fe(II), facilitate its autoxidation to Fe(III) and subsequent hydrolysis to form a ferric oxide mineral within the virion (FIG. 6D). Once again, we have observed that the nano material within the cage is both size and shape constrained by the internal dimensions of the particle. Wild type CCMV shows none of this catalytic activity and when incubated in the presence of Fe(II) all that was observed was autoxidation and hydrolysis in an uncontrolled fashion and no virion encapsulated mineral was detected. Thus, we have selectively engineered the virion to interact in a chemically specific manner which is very different from the wild type virus and which goes beyond purely electrostatic interactions.

Engineering of the N-terminal Region of the Coat Protein Subunit

We have focused our efforts on the electrostatic modifications to the N-terminal region of the coat protein subunit. From previous studies we know that the first 25 residues of the N-terminal arm are not involved in the structural integrity of the virion and are highly disordered in the crystal structure. The role of the N-terminus in the normal viral replication cycle is to package the viral RNA (Fox, J. 1997. Ph.D. Purdue University; and Fox, J. M., et al., 1994, Sem. in Vir. 5:51-60. 34) and to release the RNA during virion disassembly (Albert, F., et al., 1997, J. Virol. 71:4296-4299; and Zhao, X. 1998. Ph.D. Purdue University). We have demonstrated previously that drastic modifications to this region still allows for the in vitro assembly and isolation of empty virions (Zhao, X., et al., 1995, Virology 207:486-494; and Zhao, X., 1998, Ph.D. Purdue University). For example, the first 25 residues can be deleted with no ill effects on in vitro assembly of empty particles. Likewise, an additional 44 non-viral residues can be added to the N-terminus without compromising in vitro virion assembly. Finally, the substitution of 6 histidine residues to the N-terminus produces assembled virions. In all cases, the empty virions assembled from the altered N-terminus are structurally similar to wild type virions. Clearly, the N-terminus of the CCMV coat protein can undergo significant manipulations without preventing empty virion formation.

We are currently in the process of further manipulations to the coat protein N-terminus. We have recently completed the construction of a mutant in which all 9 basic residues (Arg, Lys) in the first 25 residues of the N-terminal arm have been replaced by glutamic acid residues. This will effectively change the entire electrostatic character of the virion interior by approximately 3240 units of charge (9×180×2) at neutral pH. Mutagenesis of this construct has been completed and the DNA has been sequenced. The mutant protein has been expressed in the E. coli expression system and soon in the P. pastoris system as well. Work is currently underway to assess the assembly of the purified coat proteins into empty virions.

Polyanionic Encapsulation in Wild Type Protein Cages

We have shown that the empty CCMV virion will uptake and encapsulate synthetic polyanions such as poly(dextran sulfate) and poly(anetholsulfonic acid) in addition to non-genomic polynucleic acid (both single and double stranded RNA and DNA) (Douglas, T., and M. J. Young, 1998, Nature 393:152-155). This interaction appears to be electrostatically driven, probably through cooperative binding which minimizes the unfavorable entropy effects.

Based on our initial experiments (see FIG. 6C) showing selective encapsulation of both poly(dextran sulfate) and poly(anetholsulfonic acid), we will investigate the range of relevant polyanion species which can be encapsulated into wild-type virions and then study their release as a function of time and environmental conditions which control the gating response. For example, suramin (Church, D., Y., et al., 1999, Cancer Chemother. Pharmacol. 43:198-204; Firsching, A., et al., 1995, Cancer Res. 55:4957-4961; Gagliardi, A. R. et al., 1998, Cancer Chemother. Pharmacol. 41:117-124; Hutson, P. R., et al., 1998, Clin. Cancer Res. 4:1429-1436; Khaled, Z., et al., 1995, Clin Cancer Res. 1:113-122; and Vassiliou, G., 1997, Eur. J. Biochem. 250:320-325), heparin (Engelberg, H., 1999, Cancer 85:257-272), the copolymer of divinyl ether-maleic anhydride, poly(acrylic acid), and the copolymer of ethylene and maleic anhydride are all therapeutically important polyanions (although not necessarily associated with any single therapeutic treatment).

We will use the commercially available polyanions: suramin, heparin (and the heparin analogs chondroitin sulfate, dermatan sulfate and mesoglycan) and the synthetic poly(acrylic acid) and poly(maleic anhydride). Briefly outlined, the empty assembled virions (0.5 mg/ml) purified from P. pastoris will be incubated with an excess of the anionic polymers at pH 7.5 (50 mM Tris) where the virions exist in their open, swollen form. Free exchange can occur between the outside and the interior of the virion. After material loading, the pH will be lowered to below the gating threshold of the virion (pH<6.5), trapping the polymer inside. This is easily achieved by rapid exchange of buffers (into 100 mM NaOAc, pH 4.5) using ultrafiltration (centricon 100). The polymer containing virion will be subsequently purified by either gradient centrifugation on 10-40% sucrose gradients or by gel filtration (medium pressure liquid chromatography). Differences in sedimentation between empty and filled virions will allow us to quantitate the virion loading. This will be compared with spectroscopic analysis (UV-Vis, 315 nm) on the previously established entrapment of poly(anetholsulfonic acid) where aromatic functionality (and associated large molar absorbtivity) allows very accurate determination of virion loading.

We will monitor the release of the polymers as a function of time by analyzing aliquots of the loaded virion by analytical centrifugation or gel filtration. By integration of peaks we can quantitate the distribution of polymer associated with the virion (high MW) and polymer which is free (low MW). Alternatively, we can employ equilibrium dialysis to spectroscopically monitor the diffusion of the individual polymers from within the virion. These time dependent release assays will also be investigated as a function of pH to determine the role played by virion gating/swelling. Controls for the uptake and release will include polymers of similar chemical composition and size but differing in charge (i.e. poly(ethylenimine) a cationic polymer, and poly(vinyl alcohol) a neutral polymer).

Small Molecule Crystallization

We propose to apply similar approaches and techniques as those used for metal anions for the crystallization of small molecules (with biomedical applications) at the protein interface within the virion. For example, we will utilize the inherently low aqueous solubility of many organic drug compounds as a means to drive the spatially selective crystallization, through control of the level of supersaturation. Encapsulation of guest molecules is clearly an entropically unfavorable event (decreased freedom) which must be offset by a favorable enthalpy (binding) of interaction. This interaction will rely in part on complementary electrostatic interactions between the drug and the protein interface as has already been shown with a number of organic and inorganic analogs. In the case of crystallization, once an initial aggregate has formed the crystal growth process is self-perpetuating because of the high affinity the molecules have for the crystal lattice and the ever increasing surface area of the growing crystal. Thus, the protein interface acts only as a nucleation catalyst by providing an interface favorable for aggregation.

As the most simple example for this model study we will first use wild-type virus and the common drug “aspirin” (acetylsalicylic acid) which carries a carboxylic acid functionality making it negatively charged at any pH close to neutral. In addition, the aromatic ring, and associated strong UV absorbance (λₘₐₓ=229 nm ε=4.8×10⁴M⁻¹cm⁻¹), allows us to monitor this molecule very easily. The solubility of acetylsalicylic acid is moderate at room temperature (saturation ˜18.5 mM) and shows a dramatic temperature dependence. Lowering the temperature even a few degrees will induce a condition of supersaturation, from which it is thermodynamically possible for crystals to form. Exploratory experiments in the absence of virion will be performed to determine the conditions for “bulk” crystallization on a reasonable timescale (i.e. a few hours). Then, the empty virions (0.5 mg/ml)will be dialyzed into a saturated solution of acetylsalicylic acid at room temperature and pH >6.5 where the virion is in its swollen conformation. The temperature of this solution will be lowered and before bulk crystallization occurs the virion will be isolated. The inner protein surface of the virion, rich in arginine and lysine residues has already been shown to induce selective crystallization of anionic molecules and we expect the virion to catalyze the crystallization of acetylsalicylic acid from slightly supersaturated solutions in a similar fashion. In addition, we will use counter ions such as Me4N+ to reduce the solubility even further and to induce crystal formation. Once the nano-crystal of the organic material has been formed the pH of the solution will be lowered to below the gating threshold (<6.5) and the crystal encapsulated virion will be isolated by gradient centrifugation or column chromatography.

Other candidate drugs and drug analogs will be tested for their ability to be crystallized within the virus protein cage. These include the antineoplastic drug diethylstilbestrol, bis-naphthalene disulfonate tetraanion (Khaled, Z., et al., 1995, Clin Cancer Res. 1:113-122), the analgesic flurbiprofen, as well as 5-fluoro-2′deoxyuridine mono(or di)phosphate. These compounds have been chosen as models because they all have a unique and sensitive analytical detection (aromatic-strong UV absorbance or F-¹⁹F NMR). Similar experimental approaches as those described for aspirin will be attempted. However, we will take advantage of the unique solubility properties of each drug to enhance the likelihood of success.

Electrostatic Modifications to the Virion Interior

Using our well established methods for site directed mutagenesis of the coat protein, we will engineer the protein so as to change the electrostatic nature of the interior protein interface while leaving structural portions of the coat protein unchanged. Thus we will change the overall charge on the interior from net positive (rich in Arg and Lys) to net negative (rich in Glu or Asp). This will allow us to broaden the range of materials which the virion can selectively entrap.

The first coat protein mutant will be the subE mutant where substitutions of the nine arginine and lysine residues for glutamic acid have been made on the non-structural N-terminus. We will continue our isolation and assembly of this virion from both E. coli and P. pastoris expression systems.

The second set of modifications will involve the complete substitution of the first 25 N-terminal amino acids with a series of varying lengths of glutamic acid-aspartic acid repeats. We have previously determined that the first 25 N-terminal amino acids can be deleted or that an additional 44 amino acids can be added, without affecting empty particle assembly. We propose to express increasing units of glutamic acid-aspartic acid repeats (2,4,6 etc. repeating units) to the N-terminus, deleted of its first 25 amino acids. Addition of the glutamic acid-aspartic acid repeats will be accomplished by PCR based site directed mutagenesis, a technique with which we are quite familiar. We will add (n+2) glutamic acid-aspartic acid repeats until addition of more repeats prevents protein cage assembly in P. pastoris and/or in vitro assembly using E. coli expressed modified coat protein. At this point we do not know how many glutamic acid-aspartic acid repeat units we will be able to add to the N-terminus, but we estimate the number to be between 5-20 repeats. In addition to adding the repeat units to the N-terminus deleted of its natural 25 amino acids, a similar series of glutamic acid-aspartic acid repeat units will be added directly to the coat protein with an intact wild type N-terminus.

The third set of mutations will involve point mutations of residues exposed on the inner surface of the protein cage, but not part of N-terminal 25 amino acids. The list of these point mutations include V34E/D, K42E/D, W47E/D. All of these point mutations will be made singlely or in combinations with each other using PCR based site-directed mutagenesis. In addition, all of these point mutations will be made in a background in which the first 25 N-terminal amino acids have been deleted. The N-terminal deletion removes 9×180=1620 positively charged residues without disrupting the protein cage structure. As an example, the K42E mutation alters the charge at the interface by 2×180=360. Thus the overall effect of this mutation (in the N-terminal deletion background) is to alter the charge on the interior surface by up to 1980 charges. We have shown that alterations at the region around residue 42 do not disrupt the structure and from the crystal structure it appears that K42 does not form salt bridges to mediate the charge. Thus the structure might be expected to also accommodate the close positioning of negative charge in the replacement of Lys by Glu (or Asp). As described below, all three series of mutations will be assayed for their ability to selectively entrap cationic therapeutic agents.

Once these mutations have been achieved and expressed in either the P. pastoris or E. coli expression system, the modified coat protein (either as assembled protein cages or as free coat protein) will be isolated by techniques well established in our laboratories (centrifugation, PEG precipitation, column chromatography). They will then be assembled into empty particles by dialysis into an assembly buffer system.

Encapsulation of Cationic Species (Polymeric and Molecular)

Assembled empty particles of the modified coat proteins expressing a negatively charged interior surface will be investigated for their ability to bind and entrap cationic and polycationic species with therapeutic relevance. Essentially the same methodologies as described above for the entrapment/crystallization and release of anionic species will be used to entrap/crystallize cationic species within the virion. The relevant polymeric species to be studied include poly(ethylenimine), poly(lysine), poly(arginine) and poly(vinylimidazoline). The relevant monomeric cationic species include simple molecules to begin with such as benzanthine dihydrochloride, benzalkonium chloride and then continue to include more complex molecules such as methotrexate HCl, tamoxifen HCl and doxirubicin HCl. The aromatic nature of the simple model compounds such as benzanthine dihydrochloride (and associated high molar absorbtivities in the UV) will be used as an efficient tool for monitoring these species both in solution and as nano-crystals packaged within the virion.

### Example 3

### Bioengineering of New Chemical Switches for the Regulated Entrapment/release of Materials

We have demonstrated that pH dependent expansion at the quasi three-fold axes is the result of deprotination of the acidic residues comprising the Ca²⁺ binding sites. The loss of protons at the elevated pH results in a close cluster of negative charges that must be accommodated either by the binding of Ca2+ or by the physical expansion (i.e. swelling) induced by electrostatic repulsion. We have taken advantage of CCMV's reversible swelling properties as a control mechanism to introduce and to release materials from the central cavity of the protein cage (see e.g. Examples 1 and 2). This reversible switching property of CCMV provides an exciting opportunity for development of elegant control mechanisms for entrapment and release of therapeutic agents.

pH Activated Chemical Switches

Gating in the wild-type virion results from electrostatic repulsion of carboxylate groups in the absence of the mediating Ca²⁺. We plan to alter the pH sense of this gating mechanism by altering the responsible carboxylate groups to histidines. Thus, the histidines are geometrically aligned for metal binding at that site and should be expected to bind well to soft metals such as Ni(II), Cu(II), and Co(II). In addition, protonation of the imidazole ring will compete with metal binding and once the metal has been lost, the close proximity of these cationic species is expected to cause a similar repulsion and opening of pores at the quasi three-fold axes. This provides a rational design for acidic switching of the gating mechanism of the virion. The mutations for this acid sensitive switch are E81H, E148H and D153H. As described above, each mutation will be generated by PCR oligonucleotide site-directed mutagenesis and introduced into both plant and P. pastoris expression systems. The empty virus particles will be and assessed for the ability to swell in response to lowered pH by changes in sedimentation velocities in 10-40% sucrose gradients (88S vs. 78S).

Redox Activated Chemical Switches

We plan to take advantage of our detailed structural knowledge of the quasi-three fold axis (the location of the Ca²⁺ binding sites) to engineer disulfide cross links between subunits (at the interface between A-B, B-C, C-A subunits in the asymmetric unit). In an oxidizing chemical environment disulfide bond formation at the quasi three-fold axis should prevent virion expansion (swelling) and thus limit entrapment or release of large molecules from the virion's interior. A change to a reducing chemical environment (like that present in the cytosol of an eukaryotic cell) breaks disulfide bonds, resulting in the expansion of the virion and the release of its entrapped molecules.

The sites that have been selected have a distance between their Ca atoms of 6.4-6.5 Å which is optimal for proper disulfide bridge formation. In general, computer modeling indicates that the lines marking the Cα to Cβ bond from each residue of the selected pairs are nearly parallel and thus close to being directly in line with one another. This leads to a 90° dihedral angle around the S-S bond that is energetically favored. The mutation pairs at the quasi-three fold axis that meet these criteria are R82C/K143C, R82C/A141C, R82C/F142C, E81C/K143C, E81C/A141C, E81C/F142C. Each set of mutations will be generated by PCR oligonucleotide site-directed mutagenesis. All mutant pairs will be confirmed by DNA sequencing and by our coupled in vitro transcription/translation assay. Each set of mutations will be introduced into both a full-length CCMV RNA 3 cDNA for expression in plant cells (when introduced into cells along with in vitro RNA transcribed with CCMV cDNAs for RNAs 1 and 2) and the P. pastoris expression system. The reduced environment of the cytosol where the empty viral particles accumulate is unlikely to facilitate disulfide bond formation. Empty virus particles expressed in P. pastoris will be purified under either oxidizing (the normal purification procedure) or under reducing conditions (in the presence of 5 mM DTT). The extent of disulfide bond formation will be assessed by mobility on SDS-PAGE gels (+/−reducing agent), quantitative reaction with Ellman's reagent (5,5′-dithiobis(2-nitrobenzoic acid)) and/or monobromobimane (quantitative assays for the presence of SH groups), as well as the ability to undergo swelling at different redox potentials (as determined by changes in sedimentation velocity on sucrose gradients).

### Example 4

Engineering Protein Cages that Express the Laminin Peptide 11 Targeting Moiety

Preliminary results already indicate that laminin peptide 11 can be expressed on the surface of the CCMV protein cage (see Results below). The proposed experiments are aimed at extending these initial results to 1) determine which of five surface exposed loops is most suitable for high-level stable expression of peptide 11, and 2) determine which of the five positions for expressing peptide 11 is most effective for in vivo targeting of cells lines expressing laminin binding protein (LBP). Briefly outlined, peptide 11 expression in P. pastioris will be analyzed in the three remaining surface loops (coat protein amino acid positions 61, 102, 161). To evaluate stable peptide 11 expression, a time course of the steady-state accumulation of empty protein cages in P. pastoris will be determined by quantitative ELISA using both CCMV antibodies specific for the assembled virion (already in use) and peptide 11 specific polyclonal antibodies (currently in production). Quantitative Western blot analysis will be used to evaluate the integrity of the coat protein-peptide 11 chimeras at each insertion site. We are also in the process of initial crystallization experiments of virions expressing peptide 11 for structural determination using X-ray crystallography.

Coat protein-peptide 11 chimeras will be analyzed for cell-targeting activity using both a cell-invasion assay and a direct competition assay for binding to cells up regulated in LBP on their cell surface. Briefly outlined, protein cages expressing peptide 11 will be tested for their ability to inhibit invasion of tumor cells through EHS basement membrane matrix. To measure invasion, a ‘Transwell’ two chamber assay system where the 8μ pore barrier is impregnated with EHS matrigel basement membrane matrix (J. R. Starkey, et al., 1999, Cytometry 35:37-47). 5×104 tumor cells are seeded into the upper well and the chambers are incubated for 3 days or one week depending on the test cell line. The upper transwells are then removed and the number of cells which have invaded into the lower well quantitated. Positive control assays with free peptide 11 and negative controls (CCMV protein cages lacking peptide 11) will be compared with CCMV protein cages expressing peptide 11.

A second independent assay will also be used to assess the cell-targeting ability of CCMV protein cages expressing peptide 11. The second assay is a competitive binding assay for cells up regulated in laminin binding protein expression. Briefly outlined, an engineered DG44CHO variant cell line with up regulated LBP expression on its surface will be used (J. R. Starkey, et al., 1999, Cytometry 35:37-47). A peptide 11 based photoprobe can be used to directly image the specific binding of peptide 11 to the surface of DG44CHO cells by confocal microscopy. This analog is biotinylated allowing for detection and quantitation using FITC-avidin. In addition, FACScan can also be used to quantitatively follow binding of peptide 11 to DG44CHO cells. Alternatively, Scatchard analysis could be utilized with ¹²⁵I-labeled peptide 11. We propose to take advantage of these well developed assays to determine if CCMV protein cages expressing peptide 11 will compete with the free peptide 11 based photoprobe for binding to DG44CHO cells. Increasing concentrations of the CCMV protein cage expressing peptide 11 will be added to a fixed amount of free peptide 11 based photoprobe and qualitatively assayed for inhibition of the photoprobe using confocal microscopy. More quantitative assays will be carried out by FACScan analysis. A corresponding analysis will also be carried out where the amount of the CCMV protein cage expressing peptide 11 will be held constant and the amount of the free peptide 11 based photoprobe will be varied. The positive control in these experiments will be the free peptide 11 lacking the photoprobe. The negative control will be CCMV protein cages lacking peptide 11.

Delivery and release of entrapped therapeutic agents at the site of attachment will be examined. For example, CCMV protein cages expressing peptide 11 demonstrating the highest affinity for LBP on DG44CHO cells will be loaded with entrapped/crystallized cytotoxic therapeutic agents. Initial studies will be performed with entrapment of suramin which is know to have cytotoxic effects on tumor cells in culture (Church, D., et al., 1999, Cancer Chemother. Pharmacol. 43:198-204). The polyanion will be entrapped as described above (see Examples 2 and 3) in protein cages where the switching mechanism is under either pH or redox control (see Example 3). After attachment to DG44CHO cells, the pH or redox environment will be changed to favor opening of the protein cage to release the entrapped material. In the case of pH mediated switching, the pH of the medium will be increased to >6.5. In the case of redox controlled gating, the medium will be reduced by the addition of DTT. Cell death will be determined by addition of viability stains (tryptophan blue) and counting both viable and non-viable cells. In addition, quantitative MTT dye reduction assays will be performed. A varying range of the polymer loaded protein cages will be attached to DG44CHO cells to determine if there is a correlation between the number of particles attached and the cell death. The appropriate negative controls of free peptide 11 alone, CCMV protein cages loaded with the polymer but lacking peptide 11, and CCMV protein cages expressing peptide 11 but not loaded with entrapped/crystallized materials will be included in all assays. This general approach will also be used to release entrapped/crystallized cationic therapeutic agents (for example methotrexate HCl, doxirubicin HCl, and tamoxifen HCl) from protein cages with modified internal electrostatic surfaces.

Results

We have recently been successful at expressing a potential cell targeting and therapeutic agent on the surface of CCMV particles. Peptide 11 region of laminin has been successfully expressed on the surface of CCMV particles. The first step was the creation of plasmid-based vectors with general utility for cloning of DNA sequences encoding for heterologous proteins as fusion proteins into the surface exposed loops of CCMV. This was accomplished by performing PCR oligonucleotide-directed mutagenesis to introduce a unique BamH1 restriction site into the regions of the CCMV coat protein cDNA corresponding to each of the five surface exposed loops (βB-βC, βD-βE, βF-βG, βC-αCD1, βH-βI. These unique, in frame, BamH1 sites were introduced at coordinates corresponding to amino acid positions 61, 102, 114, 129, and 161 in plasmid backgrounds for expression in in vivo. Expression of these BamH1 constructs, either in plant cell culture, or in the P. pastoris expression system, results in coat protein accumulation at the level similar to wild type coat protein controls. In the second step, an oligonucleotide encoding for peptide 11 (CDPGYIGSRC) with engineered Barn H1 ends was cloned into the each of the BamH1 sites corresponding to the five CCMV surface loops (βB-βC, βD-βE, βF-βG, βC-αCD1, βH-βI. Each of the peptide 11 constructs was confirmed by a coupled in vitro transcription/translation assay for full-length coat protein production and by DNA sequencing. All five constructs are currently being expressed in the P. pastoris system. To date, two of the constructs (Pep11-114 and Pep11-129; inserts into the βF-βG and βC-βCD1 loops respectively) have been initially evaluated. Expression in P. pastoris results in production of empty particles expressing peptide 11 at near wild type control levels (FIG. 4). Western blot analysis using CCMV specific antibodies demonstrate that the coat protein mobility is slightly altered due to the additional 1 kDa of peptide 11 sequence and it is intact (in contrast to other coat protein based presentation systems where the coat protein is often proteolytically cleaved at the site of foreign protein expression). We are presently re-sequencing the coat protein DNA from expressing lines to confirm that peptide 11 sequence is still present. We are currently producing a peptide 11 specific antibody to directly confirm and localize the expression of peptide 11 on the surface of the empty virus particles. We expect to begin analysis of the other three peptide 11 constructs, inserted into the remaining three surface loops expressed in P. pastoris, in the near future.

### Example 5

### Heterologous Expression Systems for Production of Viral Protein Cages

We have recently established a yeast-based heterologous protein expression system for the large scale production of modified CCMV protein cages. This is a major technical advance for our system, since it allows us to produce large quantities of protein cages independent of other viral functions (i.e. virus replication and movement). We have previously reported on the development of an E. col-based CCMV coat protein expression system (Zhao, X., et al., 1995, Virology 207:486-494). Using this system, denatured coat protein can be purified to 90% homogeneity, renatured, and assembled into empty particles which are indistinguishable from native particles (Fox, J. M., et al., 1998, Virology 244:212-218; and Zhao, X., et al., 1995, Virology 207:486-494). Unfortunately, the yields are low and the purification/in vitro assembly procedure is time consuming. In an effort to circumvent the limitations of the E. coli system, we have recently developed a second expression system based on Pichia pastoris. A full-length CCMV coat protein gene has been cloned into the pPICZ shuttle vector (InVitrogen Inc.) and integrated into the P. pastoris genome. Expression of the coat protein is under the control of a strong methanol inducible promoter (the AOX1 promoter). Methanol induction results in the high level expression of the coat protein that self-assembles into empty virus particles within P. pastoris. TEM analysis indicates that the empty virus particles are identical to native virus particles. The empty particles are efficiently purified to >99% homogeneity by lysis of P. pastoris, selective PEG precipitation of the empty particles, followed by purification on 10-40% sucrose gradients. The isolated particles were confirmed to be empty by their sedimentation velocity (50S vs. 83S for full particles), UV spectroscopy characteristics (A260/A280=0.98), and TEM staining characteristics (UA stain intrusion). Typical yields range from 1-2 mg/g FW cells. We are currently optimizing conditions for large-scale fermentation production which should dramatically increase our production of protein cages. We have already demonstrated that the empty protein cages isolated from P. pastoris can be used as constrained reaction vessels. The formation of the ferric oxide mineral within the virion (described above) was performed in N-terminal histidine modified empty protein cages isolated from P. pastoris. In addition, we have preliminary data that modified protein cages expressing peptide 11 on the particle surface can be efficiently assembled in the P. pastoris system (described above).

We claim:

1. A delivery agent comprising a protein cage with at least one entrapped medical imaging agent.

2. An agent according to claim 1, wherein said protein cage is a modified protein cage.

3. A agent according to claim 1 or 2, wherein said protein cage is a virial protein cage.

4. An agent according to claim 1 or 2, wherein said protein cage is a non-viral protein cage selected from the group consisting of ferritin, apoferritin, and dodecameric cages.

5. An agent according to claim 1 or 2, wherein said medical imaging agent is selected from the group comprising magnetic resonance imaging (MRI) agents, nuclear magnetic resonance imaging agents (NMR), x-ray agents, optical agents, ultrasound agents and neutron capture therapy agents.

6. An agent according to claim 5, wherein said imaging agent is an MRI agent comprising a paramagnetic metal ion.

7. An agent according to claim 7, wherein said paramagnetic metal ion is gadolinium III (Gd+3).

8. An agent according to claim 6 or 7, wherein said MRI agent further comprises a chelate.

9. An agent according to claim 8, wherein said delivery agent further comprises a therapeutically active agent.

10. An agent according to claim 9, wherein said therapeutically active agent is linked to said imaging agent.

11. An agent according to claim 9, wherein said therapeutically active agent is the laminin peptide 11.

12. An agent according to claim 1, 2, or 9 wherein said delivery agent comprises a second imaging agent.

13. An agent according to claim 12, wherein said second imaging agent is different from said first medical imaging agent.

14. An agent according to claim 2, wherein said protein cage is modified such that the electrostatic environment of the interior surface of said protein cage is altered.

15. A agent according to claim 14, wherein said protein coat is modified by adding at least one glutamic acid-aspartic acid repeat.

16. An agent according to claim 2, wherein said delivery agent further comprises a targeting moiety.

17. An agent according to claim 16, wherein said targeting moiety is laminin peptide 11.

18. A method of making a delivery agent comprising:
a) providing an empty protein cage and at least one medical imaging agent;
b) turning on a chemical switch to allow entry of said medical imaging agent; and,
c) turning off said chemical switch to entrap said medical imaging agent.

19. A method of making a delivery agent according to claim 18, further comprising providing at least one therapeutic agent.

20. A method of making a delivery agent according to claim 18, wherein said protein cage further comprises a targeting moiety.

21. A method of making a delivery agent according to claim 18, wherein said chemical switch is altered by the addition of cysteine residues.

22. A method of making a delivery agent according to claim 18, wherein said chemical switch is altered by the addition of histidine residues.

23. A method of making a delivery agent according to claim 18, wherein said medical imaging agent comprises an attachment linker.

24. A method according to claim 23, wherein said attachment linker is maleimide.

25. A method according to claim 23, wherein said attachment linker is a polymer.

26. A method of making a delivery agent according to claim 18, wherein said therapeutic agent comprises an attachment linker.

27. A method of making a delivery agent according to claim 26, wherein said attachment linker is a polymer.

28. An method of making a delivery agent according to claim 26 wherein said attachment linker is maleimide.

29. A method of imaging of a cell, tissue or patient comprising administering a delivery agent according to claim 1 or 2 to a cell, tissue or patient and rendering an image of said cell, tissue or patient.

30. A method according to claim 29, wherein said imaging method is selected from the group consisting of MRI, NMR, x-ray, optical ultrasound and neutron capture therapy.

31. A method of treating a disorder associated with a therapeutically active agent active comprising administering an agent according to claim 1 or 2 to a cell, tissue or patient to effect a therapeutic effect in said cell, tissue or patient.
